SEQUENCE LISTING (16 sequences)

SEQ ID NO: 1 - protein, 298 aa, Homo sapiens
MWGACKVKVHDSLATISITLRRYLRLGATMAKSKFEYVRDFEADDTCLAHCWVVVRLDGRNFHRFAEKHNFAKPNDSRALQLMTKCAQTVMEELEDIVIAYGQSDEYSFVFKRKTNWFKRRASKFMTHVASQFASSYVFYWRDYFEDQPLLYPPGFDGRVVVYPSNQTLKDYLSWRQADCHINNLYNTVFWALIQQSGLTPVQAQGRLQGTLAADKNEILFSEFNINYNNEPPMYRKGTVLIWQKVDEVMTKEIKLPTEMEGKKMAVTRTRTKPVPLHCDIIGDAFWKEHPEILDEDS

SEQ ID NO: 2 - protein, 298 aa, Mus musculus
MWAFRTARIGSLLAATSVILRRCLRLGVAMAKSKFEYVRNFEVQDTCLPHCWVVVRLDGRNFHRFAEEHNFAKPNDSRALHLMTKCAQTVMEELEDIVIAYGQSDEYSFVFRKKSNWFKRRASKFMTLVASQFASSYVFYWRDYFEDQPLRYPPGFDGRVVLYPSNQTLKDYLSWRQADCHINNLYNTVFWALIQQSGLTPVQAQQRLKGTLTADKNEILFSEFHINYNNEPHMYRKGTVLVWQKVEEVRTQEVRLPAEMEGEKKAVARTRTRVVALNCDLIGDAFWKEHPEILAEEN

SEQ ID NO: 3 - protein, 322 aa, Gallus gallus
MLGCLRAAAAISAGRGVGCVRGRASMAKSKFEYVRDFETDDTCLPNCWIVVRLDGRNFHRFAEQHEFKKPNDDRALHLMTKCAQTVMQELEDIAIAYGQSDEYSFVFKKKSKWFKRRASKFMTHVVSQFASSYVFYWKDYFKDQQLLYPPGFDGRIVLYPSNQNLKDYLSWRQADCHINNLYNTVFWMLVQRSGLTPVQAQDRLQGTLAGDKNEILFSEFNINYNNEPLMYRKGTVLIWQKVKTSLPLSSKLNIVLYSRILAVSYVELEVNEVMTKKIKLPKESEEKEVEVTRTRTKVVPLHCDIIGEQFWEEYPEILAEDS

SEQ ID NO: 4 - protein, 277 aa, Tetraodon nigroviridis
MVVEKACILRRLLKSCLVQRPACLSTSATMAKSKFEYVRNFETDDSCLRNCYIVVRLDGRNFHKFAEHHTFLKPNDGRALGLMTHSARCVMAELEDVVIAYGQSDEFSFVFKKTSTLFKRRASKLMTHVVSQFSSSYVFYWREFFGDQPLLYPPGFDGRVVLYPSNRNLRDYLSWRQADSATEENSCEGTLAADKNEILFSEFNVNYNNESAVHRKGTILIWERQEETVKKRTKSPREEKETDATVTRSRRRVQEHHCDIIGEQFWQEHPDILEQDG

SEQ ID NO: 5 - protein, 314 aa, Anopheles gambiae
VDFLQNRNRTGMQFSFPSILKHILKQTARSLHSSTMALSRFEYVKQFEQEEKLLPNSWIVVRIDGKGFHRFCNVHSFSKPNDLDALQLMNLAGMTVLQEFNEIAIGYGQSDEYSFVFRREASVYQRRRDKLVSYVASLFTSAYMFHWKRIFDGRSIAMRYPPSFDARAVLYPTDENLRDYLSWRQADVHVNNLYNTTFWNLVASGLSNSDAEKRLQGTLASDKNEILFSQFGINYNNEPIIYRKGTILLPKSVVSSEGKKQRLIVPIFEDLISDAFWAKHPEILDKKAKTDELYDLGENEAQPVVQYQLEMQER

SEQ ID NO: 6 - protein, 26 aa, Homo sapiens
MWGACKVKVHDSLATISITLRRYLRL

SEQ ID NO: 7 - protein, 279 aa, Artificial Sequence
  Description of Artificial Sequence Synthetic polypeptide
MWGACKVYLRLGATMAKSKFEYVRDFEADDTCLAHCWVVVRLDGRNFHRFAEKHNFAKPNDSRALQLMTKCAQTVMEELEDIVIAYGQSDEYSFVFKRKTNWFKRRASKFMTHVASQFASSYVFYWRFEDQPLLYPPGFDGRVVVYPSNQTLKDYLSWRQADCHINNLYNTVFWALIQQSGLTPVQAQGRLQGTLAADKNEILFSEFNINYNNELPMYRKGTVLIWQKVDEVMTKEIKLPTEMEGKKMAVTRTTPVPLHCDIIGDAFWKEHPEILDEDS

SEQ ID NO: 8 - protein, 266 aa, Artificial Sequence
  Description of Artificial Sequence Synthetic polypeptide
MWGACKVYLRLGATADDTCLAHCWVVVRLDGRNFHRFAEKHNFAKPNDSRALQLMTKCAQTVMEELEDIVIAYGQSDEYSFVFKRKTNWFKRRASKFMTHVASQFASSYVFYWRDYFEDQPLLYPPGGRVVVYPSNQTLKDYLSWRQADCHINNLYNTVFWALIQQSGLTPVQAQGRLQGTLAADKNEILFSEFNINYNNEPPMYRKGTVLIWQKVDEVMTKEIKLPTEMEGKKMAVTRTRTKPVPLHCDIIGAWKEHPEILDEDS

SEQ ID NO: 9 - protein, 271 aa, Artificial Sequence
  Description of Artificial Sequence Synthetic polypeptide
MWGACKVYLRLGATMAKSKFEYVRDFEADDTCLAHCRNFHRFAEKHNFAKPNDSRALQLMTKCAQTVMEELEDIVIAYGQSDEYSFVFKRKTNWFKRRASKFMTHVASQFASSYVFYWRDYFEDQPLPPGFDGRVVVYPSNQTLKDYLSWRQADCHINNLYNTVFWALIQQSGLTPVQAQGRLQGTLAADKNEILFSEFNINYNNEPPMYRKGTVLIWQKVDEVMTKEIKLPTEMEGKKMAVTRTRTKPVPLHDIGDAFWKEHPEILDEDS

SEQ ID NO: 10 - protein, 275 aa, Artificial Sequence
  Description of Artificial Sequence Synthetic polypeptide
MWGACKVYLRLGATMAKSKFEYVRDFEADDTCLAHCWVVVRLDGRNFHRFAEKHNNDSRALQLMTKCAQTVMEELEDIVIAYGQSDEYSFVFKRKTNWFKRRASKFMTHVASQFASSYVFYWRDYFEPLLYPPGFDGRVVVYPSNQTLKDYLSWRQADCHINNLYNTVFWALIQQSGLTPVQAQGRLQGTLAADKNEILFSEFNINYNNEPPMYRKGTVLIWQKVDEVMTKEIKLPTEMEGKKMAVTRTRTKPPHCDIIGDAFWKEHPEILDEDS

SEQ ID NO: 11 - protein, 276 aa, Artificial Sequence
  Description of Artificial Sequence Synthetic polypeptide
MWGACKVYLRLGATMAKSKFEYVRDFEADDTCLAHCWVVVRLDGRNFHRFAEKHNFAKPNDSRALQLMTKCAQTVMEELEDIVIAYGQYSFVFKRKTNWFKRRASKFMTHVASQFASSYVFYWRDYFQPLLYPPGFDGRVVVYPSNQTLKDYLSWRQADCHINNLYNTVFWALIQQSGLTPVQAQGRLQGTLAADKNEILFSEFNINYNNEPPMYRKGTVLIWQKVDEVMTKEIKLPTEMEGKKMAVTRTRTKVLHCDIIGDAFWKEHPEILDEDS

SEQ ID NO: 12 - protein, 270 aa, Artificial Sequence
  Description of Artificial Sequence Synthetic polypeptide
MWGACKVLRLGATMAKSKFEYVRDFEADDTCLAHCWVVVRLDGRNFHRFAEKHNFAKPNDSRALQLMTKCAQTVMEELEDIVIAYGQSDEYSFVFKRKTNWFKRRASKFMTHVASQFASSYVFYWRDEDQPLLYPPGPSNQTLKDYLSWRQADCHINNLYNTVFWALIQQSGLTPVQAQGRLQGTLAADKNEILFSEFNINYNNEPPMYRKGTVLIWQKVDEVMTKEIKLPTEMEGKKMAVTRTRTKPVPLHCIGDAFWKEHPEILDEDS

SEQ ID NO: 13 - protein, 272 aa, Artificial Sequence
  Description of Artificial Sequence Synthetic polypeptide
MWGACKVYLRLGATMAKSKFEYVRDFEADDTCLAHCWVVVRLDGRNFHRFAEKHNFAKPNDSRALQLMTKCAQTVMEELEDIVIAYGQSDEYSFVFKRKTNWFKRRASKFMTHVASQFASSYVFYWRFEDQPLLYPPGFDGRVVVYPSNQTADCHINNLYNTVFWALIQQSGLTPVQAQGRLQGTLAADKNEILFSEFNINYNNEPPMYRKGTVLIWQKVDEVMTKEIKLPTEMEGKKMAVTRTRTKPVPLHCDIIGDAFWKEHPEILDEDS

SEQ ID NO: 14 - protein, 263 aa, Artificial Sequence
  Description of Artificial Sequence Synthetic polypeptide
MWGACKYLRLGATMAKSKFEYVRDFEADDTCLAHCWVVVRLDGRNFHRFAEKHNFAKPNDSRALQLMTKCAQTVMEELEDIVIAYGQSDEYSFVFKRKTNWFKRRASKFMTHVASQFASSYVFYWRDEDQPLLYPPGFDGRVVVYPSNQTLKDYLSWRQADCHINNLYNTVFWALIQQSGLTPVQAQGRLQGTLAADKPPMYRKGTVLIWQKVDEVMTKEIKLPTEMEGKKMAVTRTRTKPVPLHCDIIGDAFKHPEILDEDS

SEQ ID NO: 15 - DNA, 96 bp, Artificial Sequence
  Description of Artificial Sequence Synthetic oligonucleotide
gctgttgacagtgagcgaccagtgcccttgcactgcgattagtgaagccacagatgtaatcgcagtgcaagggcactggctgcctactgcctcgga

SEQ ID NO: 16 - DNA, 97 bp, Artificial Sequence
  Description of Artificial Sequence Synthetic oligonucleotide
tgctgttgacagtgagcgccctctttctaagaagaaacattagtgaagccacagatgtaatgtttcttcttagaaagaggttgcctactgcctcgga

(19) United States
(12) Patent Application Publication
(10) Pub. No.: US 2014/0213528 A1
(43) Pub. Date: Jul. 31, 2014

(54) Fibrosuppressant Biotherapeutics

(71) Applicant: University College Dublin, National University of Ireland, Dublin (IE)

(72) Inventors: Catherine Godson, Dublin (IE); Madeline Murphy, Dublin (IE); Finian Martin, Dublin (IE); Victoria McEaneney, Dublin (IE)

(73) Assignee: University College Dublin, National University of Ireland, Dublin (IE)

(21) Appl. No.: 14/168,900

(22) Filed: Jan. 30, 2014

Related U.S. Application Data
(62) Division of application No. 12/789,304, filed on May 27, 2010.
(60) Provisional application No. 61/181,615, filed on May 27, 2009.

(51) Int. Cl. C07K 14/47; G01N 33/50; C12Q 1/68

(52) U.S. Cl. CPC C07K 14/47; C12Q 1/6809; C12Q 1/6897; G01N 33/5041
USPC 514/19.3; 514/21.2; 514/44 R; 530/350; 435/320.1; 435/6.11; 435/6.13

(57) ABSTRACT

The invention relates to IHG-1 (induced by high glucose-1) a novel gene which encodes a protein that amplifies fibrotic responses in in vitro and in vivo models of fibrotic disorders and in human diabetic nephropathy. In particular the invention relates to modifications of the IHG-1 structure which are potential fibrosuppressant biotherapeutics and modify cellular invasiveness. The invention also relates to a method of screening a therapeutic agent for suitability for the treatment of fibrotic disease comprising testing a candidate therapeutic agent for the ability to reduce the expression of IHG-1 levels in a model system.

### CROSS-REFERENCE TO RELATED APPLICATIONS

This application is a divisional of U.S. patent application Ser. No. 12/789,304, filed May 27, 2010, which in turn claims the benefit under 35 U.S.C. §119(e) of U.S. Provisional Application No. 61/181,615, filed May 27, 2009, the content of each which is hereby incorporated by reference into the present disclosure.

### FIELD OF THE INVENTION

The invention relates to IHG-1 (induced by high glucose-1) a novel gene which encodes a protein that amplifies fibrotic responses in in vitro and in vivo models of fibrotic disorders and in human diabetic nephropathy. In particular the invention relates to modifications of the IHG-1 structure, or inhibitors of IHG-1 expression, which are potential fibrosuppressant biotherapeutics and which may also find use in methods of reduction of cell motility and invasiveness, in the treatment of diseases mediated by TGF-β1 and Notch receptor activation. The invention also relates to a method of screening a therapeutic agent for suitability for the treatment of fibrotic disease comprising testing a candidate therapeutic agent for the ability to reduce the expression or activity of IHG-1 in a model system, to methods of using IHG-1 mutants to reduce hepatic gluconeogenesis and to methods of using SNPs (Single-nucleotide polymorphisms) to diagnose susceptibility to invasive cancers, arthritis and diabetic nephrophy.

### BACKGROUND TO THE INVENTION

Diabetic nephropathy (DN) is a leading cause of kidney disease, accounting for more than one third of all new cases of end-stage renal failure in Western society.^{1,2} In DN, glomerulosclerosis precedes and primes for progressive accumulation of extracellular matrix in the interstitial space, resulting in the development of tubulointerstitial fibrosis (TIF).³ TIF is a final common pathway of injury in DN and other renal diseases of diverse etiology; the extent of tubular fibrosis mirrors closely loss of renal function.^{3,4}

TGF-β1 plays a key role in regulating the pathologic changes of kidney disease, resulting in the development of TIF.3-5 TGF-β1 mediates interstitial myofibroblast activation, a critical event in the pathogenesis of interstitial fibrosis, and also induces epithelial-to-mesenchymal transformation (EMT) of tubular epithelial cells into myofibroblast cells, further contributing to renal interstitial fibrogenesis.^{6,7} TGF-β1 mediates its effects principally via activation of Smad proteins.⁸⁻¹⁰ TGF-β1 receptor activation triggers phosphorylation of the receptor-regulated Smads (R-Smad) 2 and 3.⁸⁻¹⁰ Phosphorylated R-Smad proteins bind to Smad4 and accumulate in the nucleus, where they activate transcription. The inhibitory Smads (I-Smad) 6 and 7 act in a negative feedback loop to inhibit TGF-β1 activity by preventing phosphorylation and/or nuclear accumulation of R-Smad proteins.¹¹ The critical role of Smad signaling in renal fibrogenesis is demonstrated by a number of in vivo studies. Renal fibrosis did not develop in Smad3 knockout mice with streptozotocin-induced diabetes or after unilateral ureteric obstruction (UUO), an acute model of TIF.^{12,13} In addition, overexpression of Smad7 has been shown to protect against kidney fibrosis in a number of animal models, including DN¹⁴ and UUO.¹⁵ TGF-β1 activates the Notch pathway via the notch ligand Jagged-1 in kidney disease [diabetic nephropathy, focal and segmental glomerulosclerosis] Inhibition of Notch reverses kidney failure (Kretzler and Allred 2008).

We report that induced in high glucose-1 (IHG1), a novel, highly conserved transcript, is associated with DN and UUO. Overexpression of IHG-1 amplifies TGF-β1-induced transcriptional activation in kidney tubule cells and enhances Smad3 phosphorylation. Inhibition of endogenous IHG-1 expression suppresses transcriptional responses to TGF-β1 and Smad3 phosphorylation. These data suggest that increased IHG-1 levels are likely to contribute to the TGF-β1—induced profibrotic changes in tubular cells that prime for TIF.

### OBJECT OF THE INVENTION

There are currently no therapies on the market that have been shown to arrest or reverse fibrosis. Fibrosis is the end point of many chronic inflammatory conditions leading to excessive scarring and eventual organ failure. Irrespective of the initial insult (e.g. infections, metabolic, physical) the failure of homeostatic responses to resolve injury can result in fibrosis which remains a relatively intractable condition. There is an urgent need to devise antifibrotic or/fibrosuppressant therapeutics. In this context pathways responsive to TGF-β1, the prototypic profibrotic cytokine, have been identified as appropriate therapeutic targets in multiple disorders such as fibrosis and cancer. It is an object of the invention to provide inhibitors of TGF-β1 activity (such as ITA-1 [inhibitor of TGF-β activation-1], small interfering RNA (siRNA) or short hairpin microRNA (shRNAmir)) and or modifications of ITA which represent a novel class of bio therapeutics for the treatment of fibrotic disorders. Fibrotic disorders include renal and lung, gastro-intestinal disorders. It is also an object to provide agents for the treatment of TGF-β1 regulated disorders such as cancers. Another object of the invention is to provide a method of screening a therapeutic agent for suitability for the treatment of fibrotic diseases or cancers or cellular invasiveness. A further object of the invention relates to detection of susceptibility to diseases driven by TGF-β1 based on polymorphisms in the IHG-1 gene.

### SUMMARY OF THE INVENTION

According to the present invention there is provided a protein comprising IHG-1 or a mutant thereof, the protein or the mutant thereof having a deleted or inactivated mitochondrial localisation signal. Suitably the protein has the amino acid sequence shown in FIG. 7 with a mutation in the region identified as mTP which results in the loss of the mitochondrial localisation signal. Any form of mutation which brings about inactivation of the signal would be suitable for use in the invention. Thus the mTP region may have a point, missense, nonsense, deletion or insertion mutation. In other words the protein of the invention may comprise a mutation in the mitochondrial localisation signal which inactivates the signal and may also have a mutation in the remaining portion of the molecule which does not materially affect the ability of the protein to suppress TGF-β1 signalling. The latter mutation may be in the conserved regions of the molecule as shown in FIG. 15. Alternatively the mutations may result in conservative substitution of amino-acids which have no effect on the function of the protein.

A deletion mutation may result in the loss of the sequence shown in FIG. 8, or a portion thereof. The protein may have a sequence selected from the group comprising the sequences shown in FIG. 16, or a sequence substantially similar thereto.

The invention also provides peptides derived from or peptidomimetics of the proteins disclosed above.

The invention also provides a recombinant vector comprising a nucleotide sequence encoding a protein as defined above. Suitable vectors include, but are not limited to, plenti6-V5-His, plenti4/TO/V5-DEST, and pcDNA6-V5-His.

The invention also provides a pharmaceutical composition comprising a protein, peptide or peptidometic as defined above, together with pharmaceutically acceptable carriers or excipients, or a recombinant vector as defined above. The pharmaceutical composition may be formulated for oral, topical, nasal or intra-venous administration or any other means. If the target of the pharmaceutical composition is on the plasma membrane (G-protein coupled receptor or ion channel) then the composition can get to the target passively via the circulation e.g. via renal circulation in a fibrotic renal disease state. In order to get the therapeutic into the cell it may be necessary to make a lipidated format, make a smaller stable peptide or preferably a non-peptide mimetic (via high throughput screening), or make an injectable nanoparticle (using dendrimers, chitosan, Poly(Lactide-co-Glycolide), or cyclodextrins) loaded with the agent and in some embodiments with a targeting motif specific for the cell type to be treated. Similar formulations would be suitable for delivery of siRNA to the cytosol.

In another aspect the invention provides a method reducing or alleviating fibrotic diseases or conditions or dysregulated cellular invasiveness such as cancers or arthritis, comprising administering to a patient in need of such treatment, a pharmaceutically effective amount of a protein, peptide or peptidomimetic as defined above or a recombinant vector as defined above.

In yet another aspect the invention provides use of a protein as defined above in a method of studying TGF-β1 signalling for the discovery of other therapeutic targets. The method could be used to specifically define Smad 3-driven TGF-β1 responses with a view to identifying novel therapeutic targets or to determine the efficacy of novel therapeutics. The invention relates to agents identified by this method.

Also provided is a method of screening a therapeutic agent for suitability for the treatment of fibrotic disease comprising testing a candidate therapeutic agent for the ability to reduce the expression of IHG-1 levels in a model system, wherein a reduction of expression of IHG-1 indicates suitability of the agent for treatment of fibrotic disease. The screening method may comprise in vitro or in vivo disease models such as epithelial cell transformation, fibroblast activation or unilateral ureteric obstruction.

Also provided is a method of screening a therapeutic agent for suitability for the treatment of diseases where TGF-β1 plays a pivotal role such as cancer comprising testing a candidate therapeutic agent for the ability to affect the expression or activity of IHG-1 levels in a model system, wherein a decrease in expression of IHG-1 indicates suitability of the agent for treatment of the cancer.

The invention also relates to a method of reducing or alleviating fibrotic disease and other diseases where TGF-β1 plays a pivotal role such as cancer comprising administration of pharmaceutically effective amount of a modulator of IHG-1. The modulator may reduce expression of IHG-1. Modulators include ITA-1, small interfering RNA (siRNA) or short hairpin microRNA (shRNAmir) specific for IHG-1.

The invention also provides use of a protein comprising IHG-1 or a mutant thereof, the protein or the mutant thereof having a deleted or inactivated mitochondrial localisation signal, in a method of treatment of a disease or condition mediated at least in part by TGF-β1 and Notch receptor activation, or in a method of reducing cell motility and/or invasiveness. Cell motility and/or invasiveness are significant factors in diseases such as cancers and arthritis.

The invention also provides a method of reducing hepatic gluconeogenesis comprising administration of a protein comprising IHG-1 or a mutant thereof, the protein or the mutant thereof having a deleted or inactivated mitochondrial localisation signal (such as ITA1) or an inhibitor of IHG-1 (such as siRNA) in an amount sufficient to decrease PGC1-alpha activity and its immediate consequences.

In another aspect the invention provides use of at least one SNP (Single-nucleotide polymorphism) of IHG-1 in a diagnostic method for identification of susceptibility to invasive cancers, arthritis or diabetic nephropathy.

### BRIEF DESCRIPTION OF THE DRAWINGS

FIGS. 1A-1C show that is an evolutionarily conserved ubiquitously expressed transcript. (A) IHG-1 encodes an evolutionarily conserved protein. The predicted coding sequence of IHG-1 (SEQ ID NO: 1) was aligned with homologous proteins from mouse (SEQ ID NO: 2), chicken (SEQ ID NO: 3), pufferfish (SEQ ID NO: 4), and mosquito (SEQ ID NO: 5). Conservation is indicated by text and background colour: 90%, red; 70%, blue; 50%, yellow. Mitochondrial targeting and cleavage sequences that were predicted by the programs Target P server and MitoProt II 1.0a4 are boxed and labeled mTP. (B) IHG-1 expression in normal human tissues. Autoradiograph of IHG-1 mRNA levels analyzed by Northern blot (FirstChoice Human Blot 1; Ambion, Austin, Tex.). The blot contained 2 μg of poly A RNA from the following human tissues: brain (1), placenta (2), skeletal muscle (3), heart (4), kidney (5), pancreas (6), liver (7), lung (8), spleen (9), and colon (10). Bands at approximately 3 and 1.4 kb were detected after hybridization with the IHG-1 probe. (C) The influence of high ambient glucose on IHG-1 mRNA levels in human mesangial cells (MC). MC were exposed to 5 mM glucose (lane 1) and 30 mM glucose (lane 2) for 7 d. (Left) Real-time analysis of IHG-1 expression. Data are presented as fold change after normalization to ribosomal RNA control. IHG-1 levels are increased approximately 29-fold after exposure to 30 mM glucose for 7 d. (Right) Autoradiograph of IHG-1 mRNA levels analyzed by Northern blot. A band of approximately 3 kb was detected after hybridization with the IHG-1 probe.

FIGS. 2A-2C show that increased expression of IHF-1 mRNA is associated with diabetic nephropathy (DN). (A) IHG-1 mRNA levels are increased in DN. RNA was isolated from tubule-rich micro-dissected renal biopsies taken from four living donors and four tumor nephrectomies (controls) and 11 patients with DN. Total RNA was reverse-transcribed into cDNA and analyzed for the expression of IHG-1 by quantitative real-time PCR (Taqman). Data are presented as fold change after normalization to ribosomal RNA control. Differences in means are significant (*P>0.05). (B) IHG-1 expression in DN. In situ hybridization: (1) Sections obtained from patients with advanced DN showed marked IHG-1 staining in the tubules; (2) IHG-1 expression in normal kidney; no staining is seen; (3) IHG-1 sense probe; (4) TGF-β1 staining in DN showing strong tubular staining; (5) Southwestern (SW) showing activated Smad3 in tubules in DN; and (6) control for Smad3 (SW). (C) EGF but not TGF-β1 increases IHG-1 mRNA levels. RNA was isolated from HK-2 cells treated with EGF (10 ng/ml) or TGF-β1 (3 ng/ml) for 48 h. Total RNA was reverse-transcribed into cDNA and analyzed for the expression of IHG-1 by quantitative real-time PCR (Taqman) Data are presented as fold change after normalization to ribosomal RNA control+/−SEM. Differences in means are significant (*P>0.05).

FIGS. 3A-3D show that IHG-1 expression is increased in rat unilateral ureteric obstruction (UUO). (A) Histologic detection of TIF in UUO using Gomorri trichrome staining (magnification, ×20) of a 10-d non-ligated (NL; i) and ligated (L; ii) rat kidney illustrating mature collagen fibrils present in the expanded tubulointerstitium (arrows in ii). (B) Collagen 1 mRNA levels are increased in UUO. RNA was isolated from rat L and NL kidneys 3 (n=6) and 10 d (n=5) after UUO. Total RNA was reverse-transcribed into cDNA and analyzed for the expression of collagen I by quantitative real-time PCR (Taqman). Data are presented as fold change after normalization to ribosomal RNA control+/−SEM. Differences in means are significant (*P>0.05). (C) IHG-1 mRNA levels are increased in UUO. Experimental analysis and presentation of results is as in B. Differences in means are significant (†P>0.005). (D) IHG-1 protein levels are increased in UUO. Protein extracts were prepared from rat L and NL kidneys 3 (n=3) and 10 d (n=3) after UUO. Shown are Western analyses of these extracts probed for IHG-1.

FIGS. 4A-4C show that IHG-1 overexpression amplifies TGF-β1 signal transduction. (A) IHG-1 enhances 3TP-Lux activity in response to TGF-β1. My 1 Lu cells were co-transfected with the 3TP-Lux reporter (A through C) and phRL-CMV, an internal control reporter driving the expression of Renilla luciferase (Promega, Madison, Wis.), with or without pcDNA6-IHG-1-V5 expression plasmid, as indicated. Firefly luciferase activity normalized to Renilla luciferase activity was determined as directed by the manufacturer (Promega). Where indicated, cells were stimulated with 5 ng/ml TGF-β1 24 h after transfection. Luciferase activity was measured 24 h later. The results shown are means+/−SEM of at least three independent experiments. IHG-1 significantly enhances TGF-β1 induced luciferase activity (*P>0.05). (B) IHG-1 enhances 3TP-Lux activity in response to TGF-β1 in HK-2 cells stably transfected with pIRESpuro3-IHG-1-V5. Stably transfected HK-2 cells were co-transfected with the 3TP-Lux reporter and phRL-CMV. Experimental analysis and presentation of results is as in A. IHG-1 significantly enhances TGF-β1-induced luciferase activity (‡P>0.0001). (C) IHG-1 enhances 3TP-lux activity in response to Smad3 stimulation. My 1 lu cells were co-transfected with p3TP-lux reporter, phRL-CMV, and combinations of pcDNA6-IHG-1-V5 and pRKS-Smad3 as indicated. Experimental analysis and presentation of results is as in A. IHG-1 significantly enhances Smad3-induced luciferase activity. IHG-1 significantly enhances Smad3-induced luciferase activity (*P>0.05)

FIGS. 5A-5C show that IHG-1 overexpression increases levels of phosphorylated Smad3, CTGF, and fibronectin. (A, top) IHG-1 expression increases cellular phospho-Smad3 levels after TGF-β1 stimulation of HK-2 cells stably transfected with pIRESpuro3-IHG-1-V5. Protein extracts were prepared from stably transfected HK-2 cells in the presence or absence of TGF-β1 (5.0 ng/ml) stimulation for the times indicated. Shown are Western analyses of these extracts probed for V5, phospho-Smad3, and Smad3. The IHG-1-V5 fusion protein was detected using a V5 antibody. Results shown are representative blots. (A, bottom) Alterations in phosphorylation for Smad3 levels were measured using densitometric analysis (n=3). Shown are percentage of densitometric units+/−SEM after normalization to total Smad3 protein. (B) IHG-1 expression increases CTGF levels after TGF-β1 stimulation. HK-2 cells were transduced by a lentivirus overexpressing IHG-1 (LLCIEP-IHG-1) and by a lentivirus with no insert (LLCIEP) for 24 h before serum starvation. Transduced cells were serum starved for 24 h before stimulation with TGF-β1. Protein extracts were prepared from transduced HK-2 cells in the presence or absence of TGF-β1 stimulation as indicated. Shown are Western analyses (left) of these extracts probed for CTGF, V5, and β-actin. The IHG-1-V5 fusion protein was detected using a V5 antibody. β-Actin was measured to control for equal loading of protein. Results shown are representative blots. Alterations in protein levels for CTGF were measured using densitometric analysis (right). IHG-1 enhanced TGF-β1-induced CTGF expression. Shown is fold change+/−SEM after normalization to β-actin (n=3). (C) IHG-1 expression increases fibronectin levels after TGF-β1 stimulation. Experimental analysis and presentation of results are as in A. Results shown are representative blots (left). Alterations in protein levels for fibronectin were measured using densitometric analysis (right). IHG-1 enhanced TGF-β1-induced fibronectin expression. Shown is fold change=1−SEM after normalization to β-actin (n=3).

FIGS. 6A-6D show that siRNA directed against MG-1 inhibits TGF-β1 activity. (A) HK-2 cells were transfected with either siRNA targeted against IHG-1 or scrambled siRNA. Total RNA was reverse-transcribed into cDNA and analyzed for the expression of IHG-1 by quantitative real-time PCR (Taqman). Data are presented as fold change after normalization to ribosomal RNA control. The results shown are means+/−SEM of at least three independent experiments. IHG-1 mRNA is significantly decreased; differences in means are significant (*P>0.05). (B) Total RNA was reverse-transcribed into cDNA and analyzed for the expression of glyceraldehyde-3-phosphate dehydrogenase (GAPDH) by quantitative real-time PCR (Taqman). Experimental analysis and presentation of results are as in A. No change in GAPDH mRNA levels was observed on transfection with IHG-1-specific siRNA. (C) HK-2 cells were co-transfected with the 3TP-Lux reporter, phRL-CMV, and either siRNA targeted against IHG-1 or scrambled siRNA. Firefly luciferase activity normalized to Renilla luciferase activity was determined as directed by the manufacturer (Promega). Where indicated, cells were stimulated with 5 ng/ml TGF-β1 24 h after transfection. Luciferase activity was measured 24 h later. The results shown are means+/−SEM of a representative experiment of three independent analyses. In all cases, siRNA directed against IHG-1 decreased TGF-β1 induction of the 3TP-lux reporter by at least 50%. (D) Loss of IHG-1 expression inhibits Smad3 phosphorylation. HK-2 cells were transfected with siRNA for IHG-1 or scrambled siRNA in serum-free medium for 24 h, before TGF-β1 stimulation. Protein extracts were prepared from these cells after TGF-β1 (5.0 ng/ml) stimulation for the times indicated. Shown are Western analyses (top) of these extracts probed for phospho-Smad3 and β-actin. β-Actin was analyzed to control for equal loading of protein. Results shown are representative blots (n=3). (Bottom) Alterations in phosphorylation for Smad3 levels were measured using densitometric analysis (n=3). Shown are percentage of densitometric units+/−SEM.

FIG. 7 shows that IHG-1 amino acid sequence contains an evolutionary conserved mitochondrial localization sequence. Mitochondrial localization signal designated by mTP consists of the first 29 amino acids of the IHG-1 coding sequence. Mitochondrial localization sequences are found in IHG-1 homologues in both other vertebrates and invertebrates (SEQ ID NOS 1-5, respectively, in order of appearance).

FIGS. 8A-8B show amino acid sequence of ITA-1. (A) The fifteen amino acid residues deleted in ITA-1 are underlined and highlighted in green (FIG. 8A discloses SEQ ID NO: 6). These fifteen amino acids are an essential part of the mitochondrial localization sequence of IHG-1 (B) amino acid sequence of ITA-1 (SEQ ID NO: 7).

FIG. 9 shows that doxycycline induces expression of V5-tagged ITA-1 in ITA-1-pLenti4/TO/V5-DEST-HK-2 cells]. ITA-1 expression is induced following doxycycline treatment in human renal tubule epithelial (HK2) cells stably transfected with ITA-1-pLenti4/TO/V5-DEST. Protein extracts were prepared from stably transfected HK-2 cells cultured in the presence or absence of doxycycline (0.5 ng/ml) for the times indicated. Shown are Western analyses of these extracts probed for V5 and β-actin. The IHG-1-V5 fusion protein was detected using a V5 antibody. Results shown are representative blots.

FIGS. 10A-10B show that IHG-1 is localized to mitochondria while ITA-1 is not mitochondrial associated. HK2 cells were stably transduced with pLenti4/TO/V5-DEST containing ITA-1 or IHG-1. Expression of V-5 tagged IHG-1 [Panel A] or ITA-1 [Panel B] was induced in HK-2 cells with doxycycline and detected by immunolocalisation using an anti-V5 antibody, mitochondria were detected by immunolocalisation using an anti-MnSOD antibody.

FIG. 11 shows that IHG-1 is localized to mitochondria while ITA-1 is not mitochondrial associated. Expression of V-5 tagged IHG-1 or ITA-1 in HK2 cells following transient transfection with IHG-1-pcDNA6V5-His or ITA-1-pcDNA6V5-His. Cytosolic and mitochondrial fractions were prepared and immunoblotted with anti-V5 and anti-MnSOD antibodies.

FIG. 12 shows that expression of ITA-1 suppresses TGF-β1-induced fibronectin expression. ITA-1 expression suppresses cellular fibronectin levels after TGF-1 stimulation of HK-2 cells stably transfected with ITA-1-pLenti4/TO/V5-DEST. (Top panel) Alterations in fibronectin levels normalised to β-actin levels were measured using densitometric analysis of western blots (n=4). (Lower panel) Protein extracts were prepared from stably transfected HK-2 cells, serum starved for 24 h in the presence or absence of doxycycline (0.5 ng/ml) following TGF-β1 (5.0 ng/ml) stimulation for the times indicated. Shown are Western analyses of these extracts probed for Fibronectin, V5 and β-actin. The IHG-1-V5 fusion protein was detected using a V5 antibody. Results shown are representative blots.

FIG. 13 shows that expression of ITA-1 suppresses TGF-β1-induced CTGF expression. ITA-1 expression suppresses cellular CTGF levels after TGF-β1 stimulation of HK-2 cells stably transfected with ITA-1-pLenti4/TO/V5-DEST. (Top panel) Alterations in CTGF levels normalised to β-actin levels were measured using densitometric analysis of western blots (n=4). (Lower panel) Protein extracts were prepared from stably transfected HK-2 cells, serum starved for 24 h in the presence or absence of doxycycline (0.5 ng/ml) following TGF-β1 (5.0 ng/ml) stimulation for the times indicated. Shown are Western analyses of these extracts probed for CTGF, V5 and β-actin. The IHG-1-V5 fusion protein was detected using a V5 antibody. Results shown are representative blots.

FIGS. 14A-14B show that ITA-1 suppresses 3TP-Lux activity in response to TGF-β1. ITA-1-pLenti4/TO/V5-DEST-HK-2 cells treated for 24 h with 0.5 ng/ml doxycycline (A) and My 1 Lu cells transfected with pcDNA6 or pcDNA6-ITA-1-V5 expression plasmids as indicated (B) were co-transfected with the 3TP-Lux reporter and phRL-CMV, an internal control reporter driving the expression of Renilla luciferase. Firefly luciferase activity normalized to Renilla luciferase activity was determined. Where indicated, cells were stimulated with 5 ng/ml TGF-β1 24 h after transfection. Luciferase activity was measured 24 h later. The results shown are means+/−SEM of at least three independent experiments.

FIG. 15 shows that IHG-1 is an evolutionary conserved protein. The predicted human amino acid sequence of IHG-1 (298) (SEQ ID NO: 1) was aligned with its putative splice variant (323) and with four other IHG-1 sequences from different species: Mouse (XP_{—}126359) (SEQ ID NO: 2), Chicken (XP_{—}414563) (SEQ ID NO: 3), Pufferfish (CAG10135) (SEQ ID NO: 4) and Mosquito (XP_{—}320758) (SEQ ID NO: 5). Conservation is indicated by text and background colour: 90%: Red 70%: Blue 50%: Yellow. Mitochondrial targeting and cleavage sequences. Boxed regions show highly conserved sequence. The sequence is the same as that shown in FIG. 1.

FIG. 16 shows sequence of ITA 2-8. Sequence of ITA 2-8 (SEQ ID NOS 8-14, respectively, in order of appearance). These sequences show deletion of the mTP sequence and of various conserved domains from IHG-1 to generate a series of modifications of IHG-1G-1 designated ITA 2-8.

FIG. 17 shows overexpression of V5-tagged ITA-1 in stable ITA-1-pLenti4/TO/V5-DEST-HK-2 cells. ITA-1 is overexpressed in human renal tubule epithelial (HK2) cells stably transfected with ITA-1-pLenti4/TO/V5-DEST. Protein extracts were prepared from lysates of ITA-1 and empty vector (EV) stably transfected HK-2 cells. Shown are Western analyses of these extracts probed for V5 and β-actin. The IHG-1-V5 fusion protein was detected using a V5 antibody. Results shown are representative blots. β-actin was used as a loading control.

FIG. 18 shows ITA-1 inhibited TGF-β1-induced fibronectin mRNA expression. HK-2 cells were stably transduced with lentivirally delivered ITA-1 overexpressing constructs or with an Empty Vector (EV) construct as control. Cells were serum starved for 24 h prior to stimulation with 5 ng/ml TGF-β1 for 24 h. Fibronectin mRNA levels were quantified by real-time PCR. Results shown are normalised to 18s rRNA and are means+/−SEM of at least three independent experiments.

FIG. 19 shows ITA-1 inhibited TGF-β1-induced CTGF mRNA expression. HK-2 cells were stably transduced with lentivirally delivered ITA-1 overexpressing constructs or with an Empty Vector (EV) construct as control. Cells were serum starved for 24 h prior to stimulation with 5 ng/ml TGF-β1 for 24 h. CTGF mRNA levels were quantified by real-time PCR. Results shown are normalised to 18s rRNA and are means+/−SEM of at least three independent experiments.

FIG. 20 shows ITA-1 inhibited TGF-β1-induced CTGF, fibronectin and Jagged-1 protein expression. Protein extracts were prepared from HK-2 cells were stably transduced with lentivirally delivered ITA-1 overexpressing constructs or with an Empty Vector (EV) construct as control. Cells were serum starved for 24 h prior to stimulation with 5 ng/ml TGF-31 for 24 h. Shown are Western analyses of these extracts probed for Fibronectin, CTGF, Jagged, V5 and β-actin. The IHG-1-V5 fusion protein was detected using a V5 antibody. Results shown are representative blots of at least three independent experiments.

FIG. 21 shows ITA-1 suppresses ARE-luc activity in response to TGF-β1. HK-2 cells transfected with pLenti6 or pLenti6-ITA-1-V5 expression plasmid as indicated were co-transfected with the ARE-luc reporter and phRL-CMV, an internal control reporter driving the expression of Renilla luciferase. Firefly luciferase activity normalized to Renilla luciferase activity was determined. Where indicated, cells were stimulated with 5 ng/ml TGF-β1 24 h after transfection. Luciferase activity was measured 24 h later. The results shown are means+/−SEM of at least three independent experiments.

FIG. 22 shows ITA-1 suppresses SBE-lux activity in response to TGF-β1. HK-2 cells transfected with pLenti6 or pLenti6-ITA-1-V5 expression plasmid as indicated were co-transfected with the SBE-lux reporter and phRL-CMV, an internal control reporter driving the expression of Renilla luciferase. Firefly luciferase activity normalized to Renilla luciferase activity was determined Where indicated, cells were stimulated with 5 ng/ml TGF-131 24 h after transfection. Luciferase activity was measured 24 h later. The results shown are means+/−SEM of at least three independent experiments.

FIG. 23 shows ITA-1 inhibits protein expression induced by overexpression of the constitutively active TGF-β type 1 receptor and the TGF-β1 signal mediators Smad 2 and Smad3. Protein extracts were prepared from stably transfected ITA-1 and empty vector HK-2 cells transduced with the constitutively active TGF-β type 1 receptor (CA) and TGF-β1 signalling mediators Smad 2 and Smad 3 or empty vector. Shown are Western analyses of these extracts probed for Fibronectin, CTGF, Jagged, V5 and GAPDH. The IHG-1-V5 fusion protein was detected using a V5 antibody. Results shown are representative blots from at least three experiments.

FIG. 24 shows ITA-1 inhibits migration in HK-2 cells treated with TGF-β1. Fluorescence microscopy of DAPI stained, serum starved HK-2 cells overexpressing either ITA-1 or empty vector (EV) control treated with 5 ng/ml TGF-β1. Cells were scratch wounded and left to migrate for 24 and 48 hours and stained with nuclear DAPI. The graph is a representative illustration of the migration of the cells showing % wound closure versus time.

FIG. 25 shows ITA-1 inhibits migration in MCF-10A epithelial cell monolayers treated with TGF-β1. Fluorescence microscopy of DAPI stained, serum starved MCF-10A cells overexpressing either ITA-1 or empty vector control treated with 5 ng/ml TGF-β1. Cells were scratch wounded and left to migrate for 24 and 48 hours and stained with nuclear DAPI.

FIGS. 26A-26B show overexpression of IHG-1 but not ITA-1 results in increased mitochondrial mass. (A) Mitochondrial mass was assessed in Hela cells stably overexpressing either IHG-1, ITA-1, or an empty vector control (EV) by flow cytometry following incubation with mitotracker red. Fold increase in mitochondrial mass was calculated from mean fluorescence intensities (MFI). ***, p<0.001; mean±SEM; n=3. (B) Cells overexpressing IHG-1 and control cells (EV) were visualised by fluorescence microscopy following incubation with mitotracker red.

FIGS. 27A-27B show IHG-1 results in increased stability of PGC-1α protein. (A) Protein extracts were prepared from stably transfected ITA-1 and empty vector Hela cells. Shown are Western analyses of these extracts probed for PGC-1α. β-actin demonstrates equal protein loading. (B) Hela cells stably overexpressing IHG-1 and control cells (EV) were treated with 2 μg/ml cycloheximide (Chx) for the indicated times. Whole cell lysates were analysed by western blotting with antibodies specific for PGC-1α. β-actin demonstrates equal protein loading. ***, p<0.001; mean±SEM; n=3 throughout.

FIGS. 28A-28D show expression levels of IHG-1 correlate with levels of key mediators of mitochondrial biogenesis. (A) NRF-1 and TFAM mRNA was quantified by real time PCR in Hela cells stably overexpressing IHG-1 and empty vector cells (control). (B) ATP synthase subunit 6 (ATP6) and cytochrome b (Cyto b) mRNA was quantified by real time PCR in cells from A. (C) NRF-1 and TFAM mRNA was quantified by real time PCR in Hela cells expressing tetracycline-inducible IHG-1-specific shRNAi (+dox) and control cells (−dox). (D) ATP6 and cytochrome b mRNA was quantified by real time PCR in cells from C. *, p<0.05; **, p<0.01; mean±SEM; n=3.

FIG. 29 shows sequence of shRNAmir (SEQ ID NOS 15-16, respectively, in order of appearance).

### DETAILED DESCRIPTION OF THE DRAWINGS

IHG-1 cDNA Assembly

Database searching was performed using BLAST.⁴⁴ Suppression subtractive hybridization analysis¹⁶⁻¹⁸ yielded a 198-bp cDNA fragment that we have called IHG-1 (Genbank accession no. AF110136). A sequence identical to UniGene cluster HS353090 that encoded a complete open-reading frame of 894 bp was generated by expressed sequence tag walking.

### Northern Blot Analysis and Real-Time (Taqman) PCR

Northern blots were performed using formaldehyde denaturation according to standard protocols. Transcript levels were determined by quantitative real-time Taqman PCR, as described previously.⁴⁶ Probe and primer sequences were Pre-Developed Assay Reagent (PDAR kit; Applied Biosystems, Foster City, Calif.).

### Human DN Kidney Biopsies

Human biopsy segments were obtained from patients after informed consent and with the approval of their local ethical committees.⁴⁷ ISH and Southwestern analyses were as described previously.^{48,49} Ureteric obstruction was performed in rats anaesthetized using isofluorane inhalation. Following laparotomy, the proximal portion of the left ureter was ligated with a 6/0 silk, the laparotomy closed and animals allowed to recover for 3 days (n=6) or 10 days (n=5). Animals were then anaesthetized and underwent a second laparotomy.

### Transfection

Stably transfected cell lines were generated with plasmid pIRESpuro3 and pIRESpuro3-IHG-1-V5 (Clontech, Paola Alto, Calif.). Transfection of HK-2 cells with siRNA (Dharmacon, Chicago, Ill.; SMARTpool reagent) was as described.⁵⁰

### Recombinant Lentivirus Production

HEK 293T cells were transfected with (pCMinsertR8.9), (pMD.2G), and LLCIEP or IHG-1-V5-LLCIEP using a calcium phosphate transfection kit (Invitrogen, Paisley, UK).

### Results

### IHG-1 is a Conserved Gene Transcript Induced by High Extracellular Glucose

IHG-1 (NCBI accession no. AF110136), identified by suppression subtractive hybridization¹⁶-18 is homologous to THG1L (Genbank no. NM_{—}017872, Unigene HS 353090).

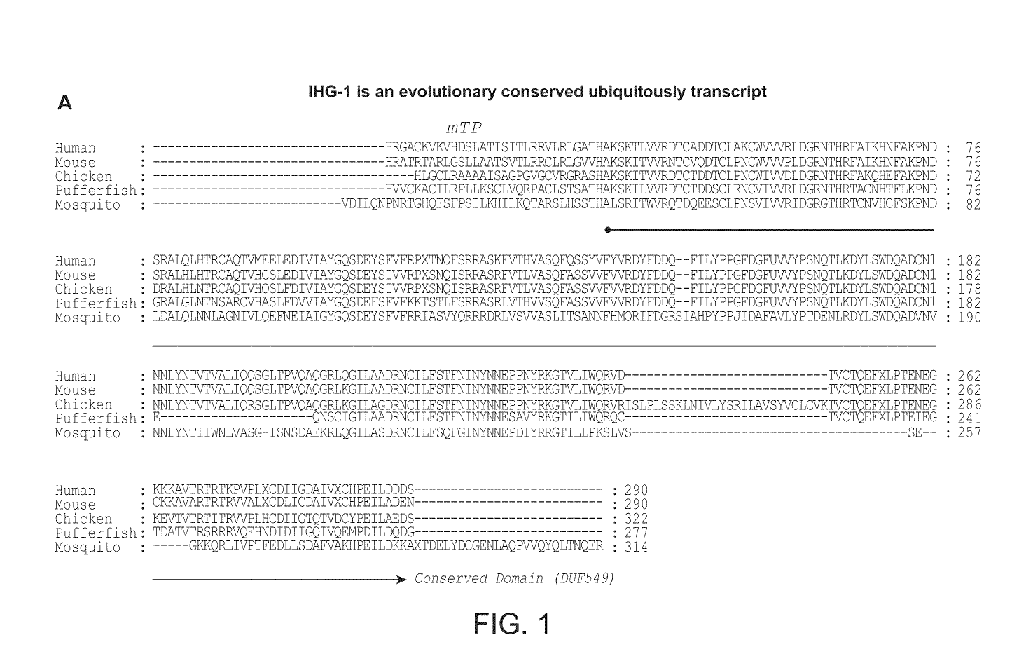

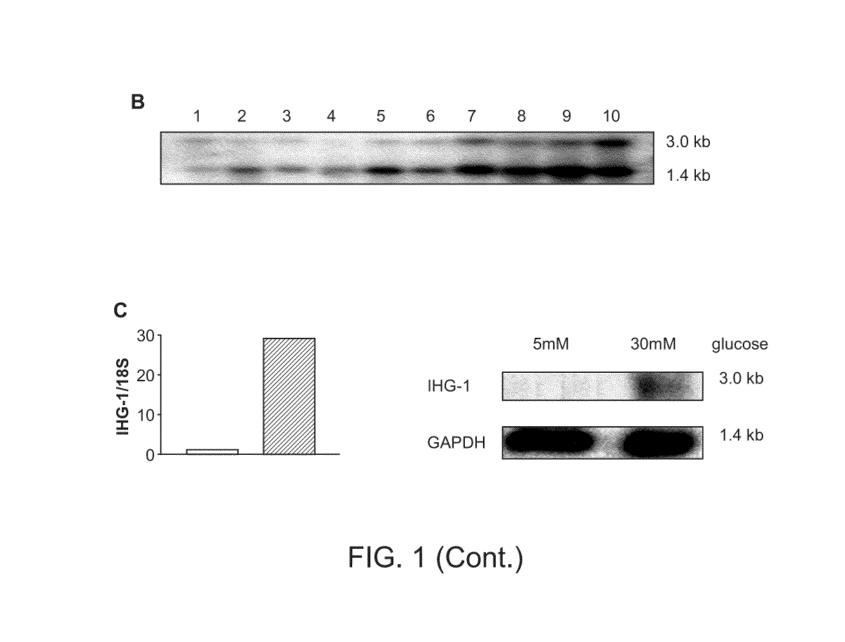

The IHG-1 amino acid sequence contains a number of regions with greater than 90% amino acid conservation between eukaryotic species (FIG. 1A). This suggests that these regions are necessary for function and therefore may represent or contain functional motifs; however, the only predicted functional domain to date is that of a mitochondrial localization signal (FIG. 1A). On discovery, therefore, IHG-1 had no known function or functional classification

Analysis by Northern blot identified two IHG-1-related transcripts of approximately 3 and 1.4 kb (FIG. 1B) in 10 human tissues, suggesting that IHG-1 was ubiquitously expressed. Although only the larger transcript (encoding IHG-1) was detected as being induced in cultured mesangial cells (MC) treated with high extracellular glucose (FIG. 1C), both transcripts are detected in normal human kidney tissue, the smaller transcript being more abundant (FIG. 1B). Induction of IHG-1 mRNA expression in primary human MC cultured in high glucose was confirmed by Northern blotting and quantitative real-time reverse transcription-PCR with primers directed against the open-reading frame (FIG. 1C).

Increased Expression of IHG-1 is Associated with Human DN

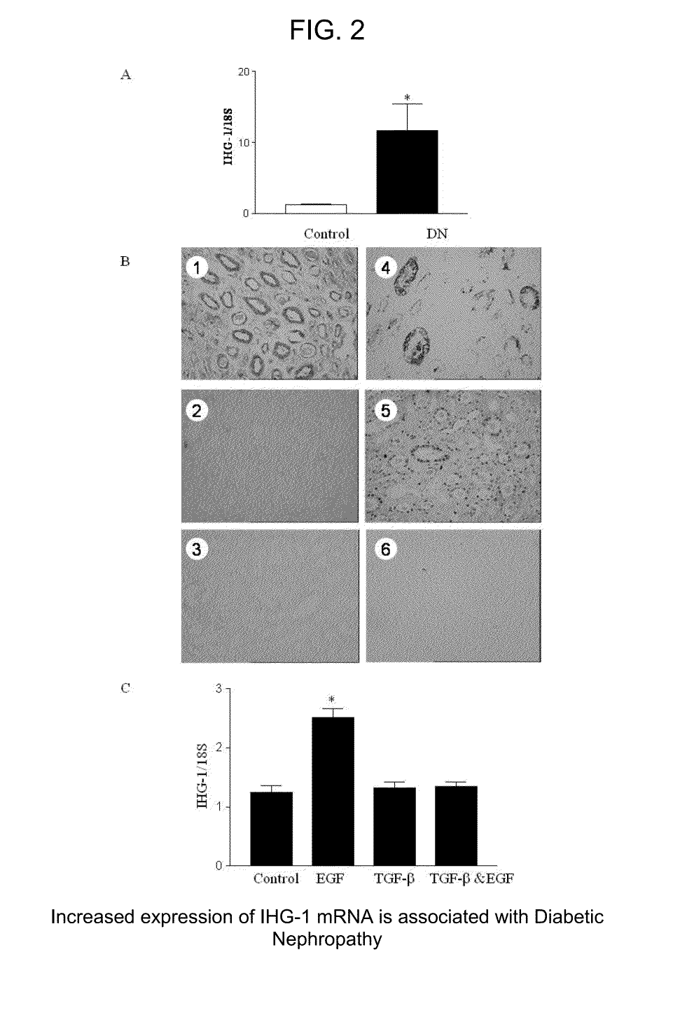

IHG-1 mRNA levels were significantly higher in tubule-rich microdissected renal biopsies from patients with DN (n=13) as compared with those taken from control kidney (mean 9.7-fold increase over control; FIG. 2A). Control tissue was from both individuals without diabetes (n=4) and from normal regions of tumor nephrectomies (n=4).

IHG-1 expression in normal kidneys and in human DN was also assessed by means of in situ hybridization (ISH). Sections hybridized with sense probes showed no staining (e.g., FIG. 2B, 3). Marked expression of IHG-1 was observed in tubular epithelial cells in biopsy specimens from patients with advanced DN (FIG. 2B, 1). TGF-β1 was also expressed abundantly in tubular epithelial cells in DN sections and seemed to have a similar pattern of expression as IHG-1 (FIG. 2B, 4). In both cases, some tubules stained more abundantly for these transcripts than others, most likely reflecting variations in cellular/regional responses to disease stress. Activated Smad3 assessed by Southwestern analysis was also observed to have a similar pattern of expression in renal tubular cells in DN (FIG. 2B, 5). Activated Smad3 was detected the in nucleus as expected. These data suggest an association between expression of this gene and tubulointerstitial change in DN. This is an area critical to disease progression and fibrosis and is associated with significant TGF-β1 presence and Smad3 function.^{21,22} In contrast to Northern analysis and reverse transcription-PCR, IHG-1 mRNA was not detected by ISH in normal kidney (FIG. 2B, 2). This is most likely due to reduced sensitivity of the ISH technique. Northern blot and reverse transcription-PCR techniques detect lower levels of mRNA than ISH, therefore IHG-1 is expressed in the normal kidney at levels that are too small to be detected by ISH.

We previously reported both treatments to increase fibronectin expression and decrease E-cadherin expression, consistent with changes associated with fibrosis.⁷ When EGF and TGF-β1 were added in combination, IHG-1 expression was not stimulated.

IHG-1 Expression is Increased in Rat Kidneys after UUO

We decided to use the UUO model of renal fibrosis to study further the role of IHG-1 in TIF. TIF is the final pathway leading to end-stage renal disease in DN and in many chronic kidney diseases.^{21,22} Because fibrosis in this model does not occur secondary to a preexisting systemic disorder, it allowed us to examine whether increased expression of IHG-1 was a feature of TIF per se.

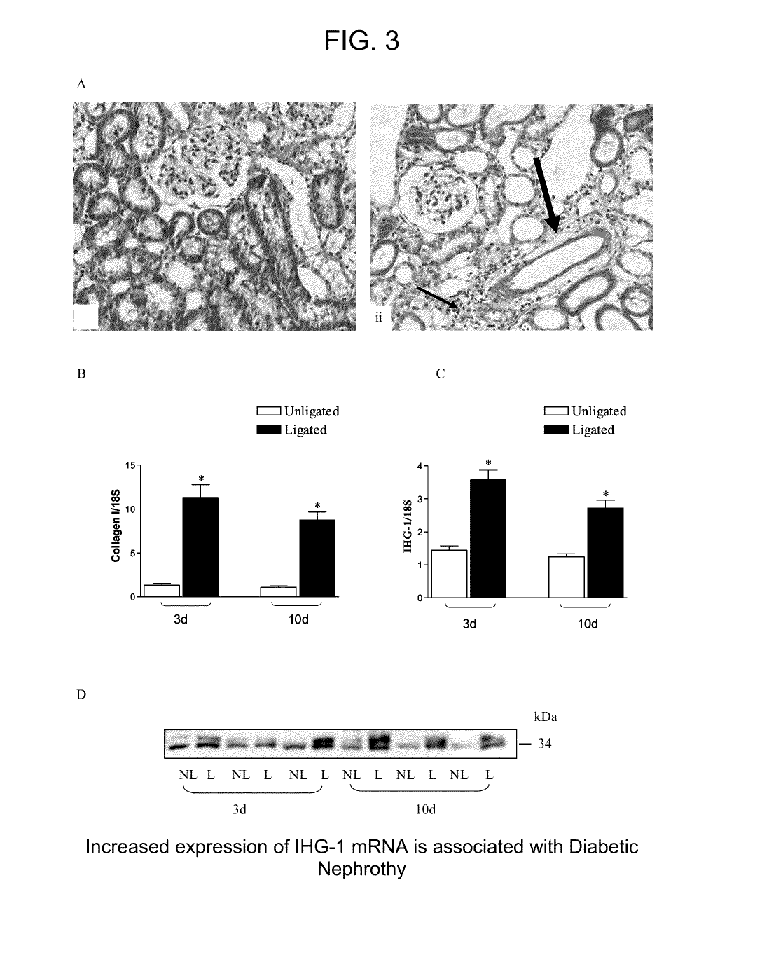

Three days after UUO, tubular dilation and interstitial inflammation was evident in the affected kidney. At 10 d after UUO, the contralateral nonligated kidneys of rats showed a staining pattern with Gomorri trichrome consistent with a normal renal morphology. The mesangial matrix and brush border of proximal tubules was clearly evident (FIG. 3A, i). In the ligated kidney, TIF was evident (thin arrow, FIG. 3A, ii) in areas of expanded and inflamed tubulointerstitium. Discrete areas of heavy collagen deposition were particularly no-table in perivascular regions (thick arrow, FIG. 3A, ii). Collagen I mRNA levels were also significantly increased in UUO both at day 3 (8.3-fold; FIG. 3B) and at day 10 after UUO (eight-fold; FIG. 3B). IHG-1 mRNA levels were significantly increased in UUO both at day 3 (2.8-fold; FIG. 3C), before development of tubulointerstitial fibrosis, and were still significantly increased at day 10 after UUO (2.2-fold; FIG. 3C), when the tubulointerstitial lesion was well advanced (FIG. 3A, ii). IHG-1 protein was increased in UUO at both day 3 and day 10 (FIG. 3D).

### IHG-1 Expression Amplifies TGF-1 Signal Transduction

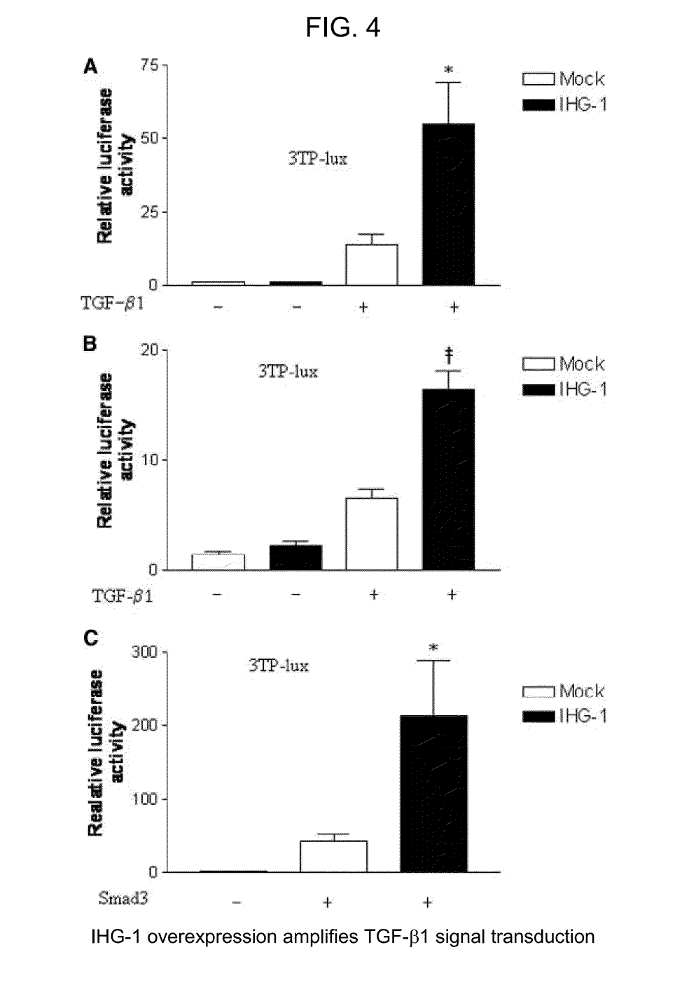

Similar to DN, activation of the TGF-β1 pathway in UUO is a pivotal event leading to development of TIF.⁵ Because IHG-1 expression was increased in kidney tubules in advanced DN and in the rat model of TIF, we examined the impact of IHG-1 overexpression on TGF-β1 signal transduction. My 1 Lu cells have been widely used to analyze transcriptional responses to TGF-β1.²⁵⁻²⁸ IHG-1 overexpression significantly enhanced TGF-β1 mediated transcription from a transfected TGF-β1-sensitive plasminogen activator inhibitor (PAI) promoter reporter construct. Levels of reporter gene expression were on average four-fold greater in cells overexpressing IHG-1 after TGF-β1 stimulation as compared with mock-transfected cells (FIG. 4A). IHG-1 overexpression had no effect on PAI promoter activity in the absence of TGF-β1 stimulation.

To investigate whether IHG-1 modulated TGF-β1 activity in a similar manner in kidney proximal tubule cells, we generated a stable cell line overexpressing IHG-1 in human renal tubular HK-2 cells. IHG-1 overexpression also increased levels of reporter gene expression after TGF-β1 stimulation in HK-2 cells and again had no effect on PAI-1 promoter activity in the absence of TGF-β1 (FIG. 4B). These data suggest that IHG-1 protein amplifies the activity of TGF-β1. Because TGF-β1 is believed to be the key mediator of TIF, these data suggest that IHG-1 may contribute to kidney disease by amplifying TGF-β1 action.

### IHG-1 Expression Prolongs Smad3 Phosphorylation and Increases Fibronectin and Connective Tissue Growth Factor Expression in Response to TGF-β1

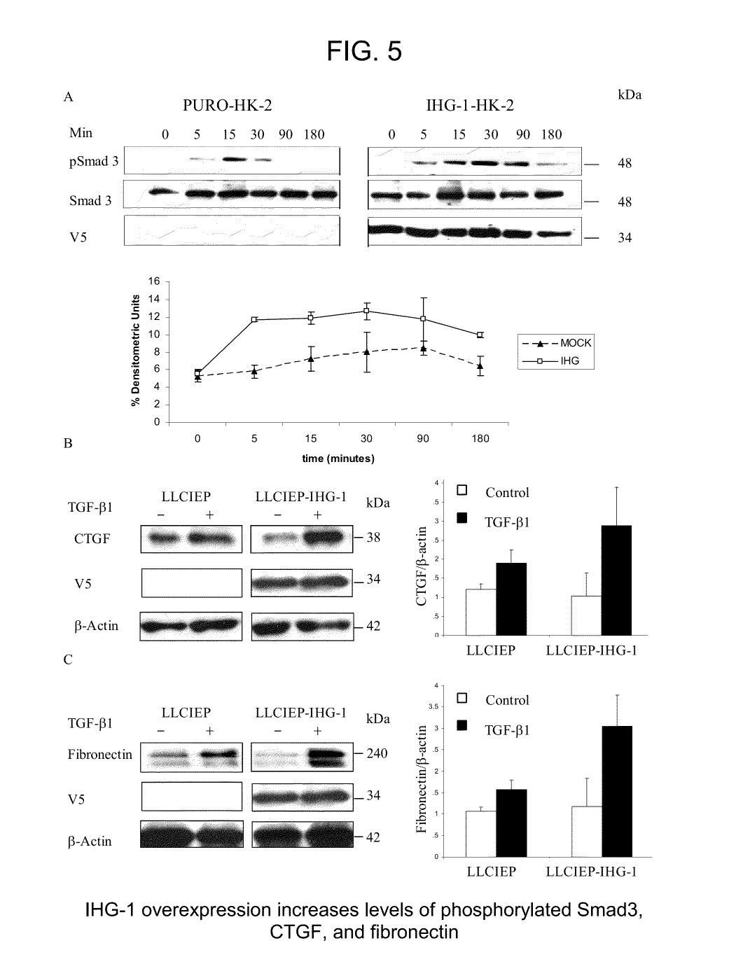

Smad-dependent responses to TGF-β1 are mediated by TGF-β1 receptor-dependent Smad2 and/or Smad3 phosphorylation.¹⁰ Overexpression of IHG-1 in stably transfected HK-2 cells resulted in increased levels of phosphorylated Smad3 after stimulation with TGF-β1 (FIG. 5A, right) as compared with mock-transfected cells (FIG. 5A, left): Higher levels of phosphorylated Smad3 were seen in the IHG-1-overexpressing cells at each time point during the 180-min response observed, whereas total Smad3 levels were unchanged by IHG-1 expression in this system (similar results were obtained in HeLa cells; data not shown). Overexpression of IHG-1 in HK-2 cells did not alter levels of pSmad2, p38, p42/44, or pAKT after stimulation with TGF-β1 (data not shown). These data suggest that IHG-1 facilitates increased levels and temporal prolongation of Smad3 phosphorylation. It is proposed that induction of the profibrotic mediator connective tissue growth factor (CTGF) by TGF-β1 is Smad3 dependent,³⁰ whereas TGF-β1-induced fibronectin expression may be both Smad dependent and Smad independent.³¹⁻³⁴ Overexpression of IHG-1 after transduction of HK-2 cells resulted in increased levels of both CTGF (2.8-fold; FIG. 5B) and fibronectin (2.6-fold; FIG. 5C) protein after stimulation with TGF-β1 as compared with mock-transduced cells (CTGF 1.56-fold, fibronectin 1.48-fold). These data suggest that IHG-1 facilitated increases and prolonged phosphorylation of Smad3, enhancing transcriptional responses to TGF-β1 along a fibrotic pathway.

### Deletion of IHG-1 Expression Inhibits TGF-β-1 Signal Transduction

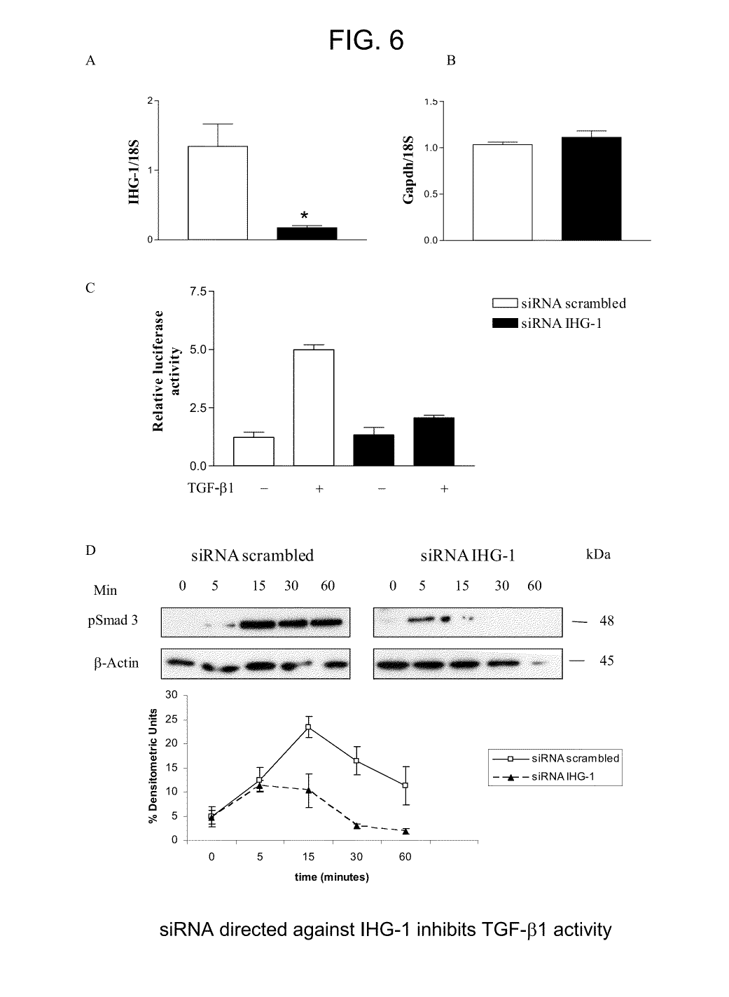

To determine whether endogenously expressed IHG-1 modulates TGF-β1 signal transduction, we used small interfering RNA (siRNA) to achieve selective knockdown of IHG-1 in HK-2 cells. IHG-1-directed siRNA (10 nM) led to an eight-fold decrease in IHG-1 expression in transfected HK-2 cells as compared with cells transfected with scrambled siRNA (FIG. 6A), whereas it had no effect on the expression of nontarget transcript glyceraldehyde-3-phosphate dehydrogenase (FIG. 6B). Transfection of IHG-1 siRNA also led to a consistent 50% decrease in PAI-luciferase reporter gene expression after TGF-β1 stimulation (FIG. 6C) and decreased levels and time of presence of phosphorylated Smad3 (FIG. 6D) after TGF-β1 stimulation, when compared with scrambled siRNA-transfected cells. These data demonstrate that endogenous IHG-1 potentially plays an active role in regulating the level of TGF-β1 signaling responses in renal tubular epithelial cells.

### Discussion

IHG-1 amplifies TGF-β1 mediated transcriptional activity by increasing and prolonging phosphorylation of Smad3 and increases TGF-β1-stimulated expression of connective tissue growth factor and fibronectin. Conversely, inhibition of endogenous IHG-1 with small interference RNA suppresses these responses to TGF-β1. ITA-1, a mutation of IHG-1 lacking the mitochondrial localization sequence, also suppresses these responses to TGF-β1.

We previously reported the identification of a novel gene transcript, IHG-1, in an in vitro screen for genes associated with development of DN.^{15,16} IHG-1 transcript levels were significantly upregulated in MC cultured in high glucose conditions, leading us to investigate whether the expression of this gene also occurred in human DN. IHG-1 transcript levels were significantly increased in tubule-rich microdissected renal biopsies from patients with DN, with clear expression being localized to the tubule in the diabetic kidney. The expression pattern was similar to that of TGF-β1 and of activated Smad3. TGF-β1 is believed to be the key mediator of fibrosis in the kidney.^{5,21,22,35} Increased activity of TGF-β1 in the tubulointerstitium resulted in increased expression and accumulation of extracellular matrix proteins, resulting in compartment-specific pathologic matrix remodeling and scarring.

In advanced DN, we hypothesized that increased IHG-1 levels are likely to contribute to the TGF-β1-induced profibrotic changes in tubular cells that prime for TIF. The significant increase in expression of IHG-1 in the UUO model adds further support to our hypothesis that IHG-1 is a mediator of TIF. UUO leads directly to TIF, in contrast to DN, in which changes in the glomeruli come first and lead to the development of the tubulointerstitial lesion. Increased expression of IHG-1 in this model of renal fibrosis suggests that this novel gene may contribute to TIF per se and may not be restricted to DN. Observations of increased IHG-1 expression in the tubules in other fibrotic diseases add further weight to our hypothesis.

Although the initiating stresses in DN and UUO are different, the development of TIF is associated with common cytokine/growth factor stimuli.⁵ For instance, both conditions have been successfully treated experimentally with bone morphogenic protein-7.^{36,37} EGF receptor activation has been implicated in tubulointerstitial fibrogenesis.⁵ It is transactivated by high extracellular glucose²⁴ and has been proposed to assist in the selective survival of a transdifferentiated, profibrotic cell type.⁷ One of the ways in which it may facilitate the profibrotic effects on tubule cells may be via induction of IHG-1 expression.

Our investigations of IHG-1's effect on responses to TGF-β1 stimulation in renal tubule cells clearly points to IHG-1's being an amplifier of TGF-β1 action in the tubule. Its over-expression-induced increases in luciferase reporter activity from a TGF-β1 responsive region of the PAI-1 promoter. PAI-1 is strongly induced in various kidney pathologies, including DN and UUO, and is considered an important factor in the development of renal fibrosis.^{21,22,38} IHG-1 had no effect on basal levels of reporter expression, suggesting that signal transduction must be first initiated for IHG-1 to mediate its effects. Activated Smad3 co-localized with IHG-1 in DN, suggesting that IHG-1 might influence TGF-β1 signaling by targeting Smad3.

TGF-β1 stimulation of epithelial cells causes a transient phosphorylation of R-Smad evident within 10 min, peaking between 30 and 60 min and persisting for up to 5 h.10,39 Both R-Smad and Smad nuclear accumulation are maintained only when receptors are active.⁸⁻¹¹ As soon as receptor activity decreases, R-Smad phosphorylation decreases and nuclear accumulation is lost. Dephosphorylation is proposed as the main mechanism of deactivation.⁸⁻¹¹ The majority of phospho-Smad are believed to be recycled after dephosphorylation, phospho-Smad3 by the phosphatase PPM1A.10,40 The early and sustained increase in Smad3 phosphorylation in epithelial cells overexpressing IHG-1 coupled with the rapid loss of phosphorylation with IHG-1 knockdown suggests that IHG-1 may function by inhibiting the activity of a phosphatase, which may target either the activated receptor (e.g., GADD34)41 or the R-Smad itself. How this may function will be the subject of our future investigations. It is also possible that IHG-1 may modulate the activity of the I-Smad, Smad7, which not only targets TGF-β1 receptors for dephosphorylation but also both receptors and R-Smad for proteosomal degradation.¹¹ The dephosphorylation machinery is believed to be active and in place from onset to termination of signaling; thereby, signal transduction and consequent gene transcription relies on a dynamic balance between phosphorylation and dephosphorylation.⁸⁻¹¹ Increased expression of IHG-1, leading to increased phosphorylation of Smad3, may tip this balance in favor of fibrosis.

Smad3 is required for TGF-β1-induced fibrosis. ^{12,13,42} An increasing body of evidence demonstrates that decreasing TGF-β1 signaling through blocking Smad3 can protect against fibrosis both in vivo and in vitro.^{12,13,38} It has been reported that renal fibrosis did not develop in Smad3 knockout mice with streptozotocin-induced diabetes or after UUO. In addition, cells from these mice failed to undergo an EMT when stimulated with TGF-β1.^{12,13} Interestingly, we detected no amplification of Smad2 activation by IHG-1. Why the effect of IHG-1 is Smad3 specific remains unknown and will be the subject of our future investigations.

Expression of CTGF, a profibrotic mediator, is increased in the tubular epithelium by TGF-β1 and is proposed to play a key role in renal fibrogenesis.⁴³ Overexpression of IHG-1 increased TGF-β1-induced CTGF expression, further strengthening our hypothesis that IHG-1 contributes to the development of TIF. IHG-1 expression also increased TGF-β1-induced fibronectin expression, which has been reported to be induced by both Smad-dependent and Smad-independent pathways.³¹⁻³⁴ Our data suggest that IHG-1 amplifies TGF-β1-induced fibronectin expression by a Smad-dependent mechanism; however, there is also the possibility that this induction is Smad dependent and indirect, mediated, for instance, by CTGF.

Suppression of IHG-1 expression using siRNA led to reduced TGF-β1 induction of the PAI promoter activation and reduced levels of Smad3 phosphorylation, suggesting that IHG-1 has an important role in promoting TGF-β1 responses in renal tubular cells, and given the ubiquitous nature of tissue IHG-1 expression, this may be a general phenomenon. Thus, we describe IHG-1, a novel protein-encoding transcript, whose increased expression is associated with renal tubular elements in human DN and in kidney tissue in the rat UUO model of interstitial fibrosis. We show, on overexpression, that IHG-1 is a novel amplifier of a TGF-β1 transcriptional response possibly through increasing and/or maintaining phosphorylated Smad3 protein levels after receptor activation by TGF-β1. In addition, knockdown of endogenous IHG-1 blunts Smad3 phosphorylation and a TGF-β1 transcriptional response. Considering TGF-β1's central role in the development of fibrotic renal disease, IHG-1 may well constitute a novel profibrotic mediator.

Expression of ITA-1 in human cells in culture has been shown to be associated with inhibition of responses to the prototypic fibrotic mediator TGFβ1. In cells expressing ITA-1, TGF-β1 stimulated fibronectin and CTGF expression was inhibited. ITA expression is associated with decreased TGF-β1-driven gene expression as demonstrated by the PAI-1 luciferase assay. Expression of ITA-1 by alternative strategies to doxycycline induction generates similar results [eg. transient transfection with plasmid constructs i.e. pcDNA6-V5-His or generation of stable overexpressing cell lines]. To further define the fibrosuppressive actions of ITA a series of constructs ITA2-8 were constructed, in each construct one of the evolutionary conserved domains was deleted. These constructs permit identification of region(s) necessary for the fibrosuppressant bioactions demonstrated for Anti-ITA-1. A truncated construct ITA9. containing domains necessary for fibrosuppressive bioactions, will allow more efficient and increased ease of delivery to cells, delivery in vivo and provide for a lead compound/peptidomimetic for high throughput screens. These constructs will further inform the design of peptides or peptidomimetics, which will be more amenable to intracellular delivery.

TGF-β1 induced migration of epithelial cells is thought to contribute towards cancer metastasis, and is a feature of EMT. ITA-1 inhibits this TGF-β1 response in both kidney and mammary epithelial cell lines. Cellular invasiveness is also associated with fibroblast activation as seen in rheumatoid arthritis.

The transcriptional coactivator PGC-1alpha plays a central role in the coordination of mitochondrial biogenesis. PGC-1alpha is a tightly regulated protein and its dysfunction has been implicated in the pathogenesis of several human diseases. Mitochondrial dysfunction is a major contributor to hyperglycaemic-induced renal damage (2). Mitochondrial cytopathies also result in a range of renal pathologies including chronic tubulo-interstitial fibrosis (TIF), multi-cystic disease and focal segmental glomerulosclerosis (3). IHG-1 overexpression causes increased mitochondrial mass and stabilisation of PGC-1αprotein. Consistent with increased mitochondrial mass we observe upregulation of PGC-1α-regulated transcription factors, including nuclear respiratory factor 1 (NRF-1) and mitochondrial transcription factor A (TFAM), a key activator of the transcription of mitochondrially encoded genes, along with increased expression of mitochondrial proteins. Conversely, inhibition of endogenous IHG-1 expression using shRNAmir resulted in reduced PGC-1αprotein, decreased expression of NRF-1 and TFAM, and reduced expression of mitochondrial proteins. A strong case can thus be made for the potential of ITA1 [and IHG-1 shRNAmir] to provide benefit in suppressing DN progression, and that of the associated fibrosis, by directly inhibiting changes in mitochondria orchestrated by PGC1alpha. In addition, hepatic gluconeogenesis is an established core contributor to hyperglycaemia in type 2 diabetes. PGC1alpha is an accepted driver of this gluconeogenesis. Again, a strong case can be made for the potential of ITA1 [and IHG-1 shRNAmir] to provide benefit in suppressing gluconeogenesis and thus hyperglycaemia, orchestrated by PGC1alpha, in type 2 diabetes.

The words “comprises/comprising” and the words “having/including” when used herein with reference to the present invention are used to specify the presence of stated features, integers, steps or components but does not preclude the presence or addition of one or more other features, integers, steps, components or groups thereof.

Throughout this disclosure, various publications, patents and published patent specifications are referenced by author and/or by citation or alternatively, by an Arabic numeral. The complete bibliographic information for the reference identified by an Arabic numeral is found at the end of the specification, immediately preceding the claims. The disclosures of all publications, patents and published patent specifications are hereby incorporated by reference into the present disclosure to more fully describe the state of the art to which this invention pertains.

The terms “polynucleotide” and “oligonucleotide” are used interchangeably and refer to a polymeric form of nucleotides of any length, either deoxyribonucleotides or ribonucleotides or analogs thereof. Unless specifically noted otherwise, a polynucleotide of this invention can be purified, isolated or recombinant. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polymer. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form.

The term “isolated” means separated from constituents, cellular and otherwise, in which the cell, tissue, polynucleotide, peptide, polypeptide, protein, antibody or fragment(s) thereof, which are normally associated in nature. For example, an isolated polynucleotide is separated from the 3′ and 5′ contiguous nucleotides with which it is normally associated in its native or natural environment, e.g., on the chromosome. As is apparent to those of skill in the art, a non-naturally occurring polynucleotide, peptide, polypeptide, protein, antibody or fragment(s) thereof, does not require “isolation” to distinguish it from its naturally occurring counterpart. An isolated cell is a cell that is separated form tissue or cells of dissimilar phenotype or genotype. Unless otherwise specified, a polypeptide or other naturally occurring product is isolated, recombinant or purified to distinguish it from naturally occurring products of nature.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

### REFERENCES

1. Excerpts from United States Renal Data System (1999) Annual Data Report. Am J Kidney Dis. 34(2 suppl 1):s1-176.
2. Parring, H. H., Osterby, R., Anderson, P. W., and Hsuch, W. A. (1996) in Brenner and Rector's The Kidney (Brenner, B. M., ed), 5th Ed., pp. 1864-1883, W. B. Saunders, Philadelphia
3. Mason, R M and Abdel Wahab, N. (2003) Extracellular matrix metabolism in diabetic nephropathy J Am Soc Nephrol 14: 1358-1373.
4. Bottinger, E. P. and Bitzer, M. (2002), TGF-beta signaling in renal disease, J Am Soc Nephrol 13: 2600-2610
5. Docherty N G, O'Sullivan O E, Healy D A, Fitzpatrick J M, Watson R W. (2006) Evidence that inhibition of tubular cell apoptosis protects against renal damage and development of fibrosis following ureteric obstruction. Am J Physiol Renal Physiol. 290(1):F4-13
6. Yang J, Liu Y. (2001) Dissection of key events in tubular epithelial to myofibroblast transition and its implications in renal interstitial fibrosis. Am J Pathol. 159(4):1465-75.
7. Docherty N G, O'Sullivan O E, Healy D A, Murphy M, O'Neil A J, Fitzpatrick J M, Watson R W. (2006) TGF-beta1-induced EMT can occur independently of its proapoptotic effects and is aided by EGF receptor activation. Am J Physiol Renal Physiol. 290(5):F1202-12.
8. Shi Y, Massague J. (2003) Mechanisms of TGF-beta signaling from cell membrane to the nucleus. Cell 113(6):685-700.
9. Moustakas A, Souchelnytskyi S, Heldin C H. (2001) Smad regulation in TGF-beta signal transduction. J Cell Sci 114(Pt 24):4359-69
10. Attisano L, Wrana J L (2002) Signal transduction by the TGF-beta superfamily. Science. 296(5573):1646-7.
11. Itoh S, ten Dijke P. (2007) Negative regulation of TGF-beta receptor/Smad signal transduction. Curr Opin Cell Biol. 19(2):176-84.
12. Sato M, Muragaki Y, Saika S, Roberts A B, Ooshima A. (2003) Targeted disruption of TGF-beta1/Smad3 signaling protects against renal tubulointerstitial fibrosis induced by unilateral ureteral obstruction. J Clin Invest 2(10):1486-94.
13. Fujimoto M, Maezawa Y, Yokote K, Joh K, Kobayashi K, Kawamura H, Nishimura M, Roberts A B, Saito Y, Mori S. (2003) Mice lacking Smad3 are protected against streptozotocin-induced diabetic glomerulopathy. Biochem Biophys Res Commun 305(4): 1002-7
14. Lund R J, Davies M R, Hruska K A. (2002) Bone morphogenetic protein-7: an anti-fibrotic morphogenetic protein with therapeutic importance in renal disease. Curr Opin Nephrol Hypertens 11(1):31-6.
15. Lan H Y, Mu W, Tomita N, Huang X R, Li J H, Zhu H J, Morishita R, Johnson R J (2003) Inhibition of renal fibrosis by gene transfer of inducible Smad7 using ultrasound-microbubble system in rat UUO model. J Am Soc Nephrol 14(6):1535-48.
16. Diatchenko L, Lau Y F, Campbell A P, Chenchik A, Moqadam F, Huang B, Lukyanov S, Lukyanov K, Gurskaya N, Sverdlov E D, et al (1996) Suppression subtractive hybridization: a method for generating differentially regulated or tissue-specific cDNA probes and libraries. Proc Natl Acad Sci USA 93(12):6025-30.
17. Murphy M, Godson C, Cannon S, Kato S, Mackenzie H S, Martin F, Brady H R. (1999) Suppression subtractive hybridization identifies high glucose levels as a stimulus for expression of connective tissue growth and other genes in human mesangial cells. J Biol Chem 274(9):5830-4.
18. Clarkson M, Murphy M, Gupta S, Lambe T, Godson C, Mackenzie H S, Martin F, Brady H R. (2002) High glucose-altered gene expression in mesangial cells: actin-regulatory protein gene expression is triggered by oxidative stress and cytoskeletal disassembly J Biol Chem 277(12):9707-12
19. Gu W, Jackman J E, Lohan A J, Gray M W, Phizicky E M. tRNAHis maturation: an essential yeast protein catalyzes addition of a guanine nucleotide to the 5′ end of tRNAHis (2003) Genes Dev 17(23):2889-901
20. Guo D, Hu K, Lei Y, Wang Y, Ma T, He D. (2004) Identification and characterization of a novel cytoplasm protein ICF45 which is involved in cell cycle regulation. J Biol Chem 279(51):53498-505.
21. Liu Y. (2006) Renal fibrosis: new insights into the pathogenesis and therapeutics. Kidney Int 69(2):213-7
22. Wang W, Koka V, Lan H Y (2005) Transforming growth factor-beta and Smad signalling in kidney diseases. Nephrology 10 (1):48-56.
23. Gilbert R E, Cox A, McNally P G, Wu L L, Dziadek M, Cooper M E, Jerums G. (1997) Increased epidermal growth factor in experimental diabetes related kidney growth in rats. Diabetologia 40(7):778-85
24. Saad S, Stevens V A, Wassef L, Poronnik P, Kelly D J, Gilbert R E, Pollock C A. (2005) High glucose transactivates the EGF receptor and up-regulates serum glucocorticoid kinase in the proximal tubule. Kidney Int 68(3):985-97.
25. Weis-Garcia F, Massagué J. (1996) Complementation between kinase-defective and activation-defective TGF-beta receptors reveals a novel form of receptor cooperativity essential for signaling. EMBO J 15(2):276-89.
26. Feng X H, Derynck R (1996) Ligand-independent activation of transforming growth factor (TGF) beta-signaling pathways by heteromeric cytoplasmic domains of TGF-beta receptors. J Biol Chem 271: 13123-13129,
27. Tsukazaki T, Chiang T A, Davison A F, Attisano L, Wrana J L. (1998) SARA, a FYVE domain protein that recruits Smad2 to the TGFbeta receptor. Cell 95(6):779-91.
28. Xu W, Angelis K, Danielpour D, Haddad M M, Bischof O, Campisi J, Stavnezer E, Medrano E E (2000) Ski acts as a co-repressor with Smad2 and Smad3 to regulate the response to type beta transforming growth factor. Proc Natl Acad Sci USA 97(11):5924-9.
29. Moustakas A, Heldin C H. (2002) From mono- to oligo-Smads: the heart of the matter in TGF-beta signal transduction. Genes Dev 16(15):1867-7
30. Phanish M K, Wahab N A, Colville-Nash P, Hendry B M, Dockrell M E. (2006) The differential role of Smad2 and Smad3 in the regulation of pro-fibrotic TGFbeta1 responses in human proximal-tubule epithelial cells. Biochem J 393(Pt 2):601-7
31. Niculescu-Duvaz I, Phanish M K, Colville-Nash P, Dockrell M E. (2007) The TGFbeta1-induced fibronectin in human renal proximal tubular epithelial cells is p38 MAP kinase dependent and Smad independent. Nephron Exp Nephrol 105(4):e108-16.
32. Li J, Campanale N V, Liang R J, Deane J A, Bertram J F, Ricardo S D (2006) Inhibition of p38 mitogen-activated protein kinase and transforming growth factor-beta1/Smad signaling pathways modulates the development of fibrosis in adriamycin-induced nephropathy. Am J Pathol 169(5):1527-40
33. Isono M, Chen S, Hong S W, Iglesias-de la Cruz M C, Ziyadeh F N. (2002) Smad pathway is activated in the diabetic mouse kidney and Smad3 mediates TGF-beta-induced fibronectin in mesangial cells. Biochem Biophys Res Commun 296(5):1356-65.
34. Li Y, Yang J, Dai C, Wu C, Liu Y. (2003) Role for integrin-linked kinase in mediating tubular epithelial to mesenchymal transition and renal interstitial fibrogenesis J Clin Invest 112(4):503-16.
35. Hoffman B B, Sharma K, Zhu Y, Ziyadeh F N. (1998) Transcriptional activation of transforming growth factor-beta1 in mesangial cell culture by high glucose concentration. Kidney Int 54(4):1107-16.
36. Hruska K A, Guo G, Wozniak M, Martin D, Miller S, Liapis H, Loveday K, Klahr S, Sampath T K, Morrissey J (2000) Osteogenic protein-1 prevents renal fibrogenesis associated with ureteral obstruction. Am J Physiol Renal Physiol 279(1):F130-43.
37. Sugimoto H, Grahovac G, Zeisberg M, Kalluri R. (2007) Renal fibrosis and glomerulosclerosis in a new mouse model of diabetic nephropathy and its regression by bone morphogenic protein-7 and advanced glycation end product inhibitors. Diabetes. 56(7):1825-33.
38. Matsuo S, Lopez-Guisa J M, Cai X, Okamura D M, Alpers C E, Bumgarner R E, Peters M A, Zhang G, Eddy A A. (2005) Multifunctionality of PAI-1 in fibrogenesis: evidence from obstructive nephropathy in PAI-1-overexpressing mice. Kidney Int 67(6):2221-38
39. Inman G J, Nicolas F J, Hill C S. (2002) Nucleocytoplasmic shuttling of Smads 2, 3, and 4 permits sensing of TGF-beta receptor activity. Mol Cell 10(2):283-94.
40. Lin X, Duan X, Liang Y Y, Su Y, Wrighton K H, Long J, Hu M, Davis C M, Wang J, Brunicardi F C, Shi Y, Chen Y G, Meng A, Feng X H. (2006) PPM1A functions as a Smad phosphatase to terminate TGFbeta signaling. Cell. 125(5):915-28.
41. Shi W, Sun C, He B, Xiong W, Shi X, Yao D, Cao X. (2004) GADD34-PP1c recruited by Smad7 dephosphorylates TGFbeta type I receptor. J Cell Biol 164(2):291-300.
42. Flanders K C. (2004) Smad3 as a mediator of the fibrotic response. Int J Exp Pathol 85(2):47-64.
43. H, Kikuta T, Kobayashi T, Inoue T, Kanno Y, Takigawa M, Sugaya T, Kopp J B, Suzuki H. (2005) Connective tissue growth factor expressed in tubular epithelium plays a pivotal role in renal fibrogenesis. J Am Soc Nephrol 16(1):133-43.
44. Altschul, S. F., Madden, T. L., Schäffer, A. A., Zhang, J., Zhang, Z., Miller, W., and Lipman, D. J. (1997) Gapped BLAST and PSI-BLAST: a new generation of protein database search programs. Nucleic Acids Res 25, 3389-3402
45. McMahon R, Murphy M, Clarkson M, Taal M, Mackenzie H S, Godson C, Martin F, Brady H R (2000) IHG-2, a mesangial cell gene induced by high glucose, is human gremlin. Regulation by extracellular glucose concentration, cyclic mechanical strain, and transforming growth factor-beta1. J Biol Chem 275(14):9901-4.
46. Crean J K, Finlay D, Murphy M, Moss C, Godson C, Martin F, Brady H R. (2002) The role of p42/44 MAPK and protein kinase B in connective tissue growth factor induced extracellular matrix protein production, cell migration, and actin cytoskeletal rearrangement in human mesangial cells. J Biol Chem 277(46):44187-94.
47. Cohen C D, Frach K, Schlondorff D, Kretzler M: (2002) Quantitative gene expression analysis in renal biopsies: a novel protocol for a high-throughput multicenter application. Kidney Int 61:133-140,
48. Mezzano S A, Droguett M A, Burgos M E, Ardiles L G, Aros C A, Caorsi I, Egido J. (2000) Overexpression of chemokines, fibrogenic cytokines, and myofibroblasts in human membranous nephropathy. Kidney Int 57 (1): 147-58.
49. Mezzano S A, Barria M, Droguett M A, Burgos M E, Ardiles L G, Flores C, Egido J. (2001) Tubular NF-kappaB and AP-1 activation in human proteinuric renal disease. Kidney Int 60(4): 1366-77.
50. Healy D A, Daly P J, Docherty N G, Murphy M, Fitzpatrick J M, Watson R W. (2006) Heat shock-induced protection of renal proximal tubular epithelial cells from cold storage and rewarming injury. J Am Soc Nephrol 17(3):805-12

What is claimed is:

1. An IHG-1 protein or a mutant protein thereof for the treatment of fibrotic disease or for the treatment of conditions characterised by dysregulated cellular invasiveness, such as cancers, the protein or mutant having a deleted or inactivated mitochondrial localisation signal.

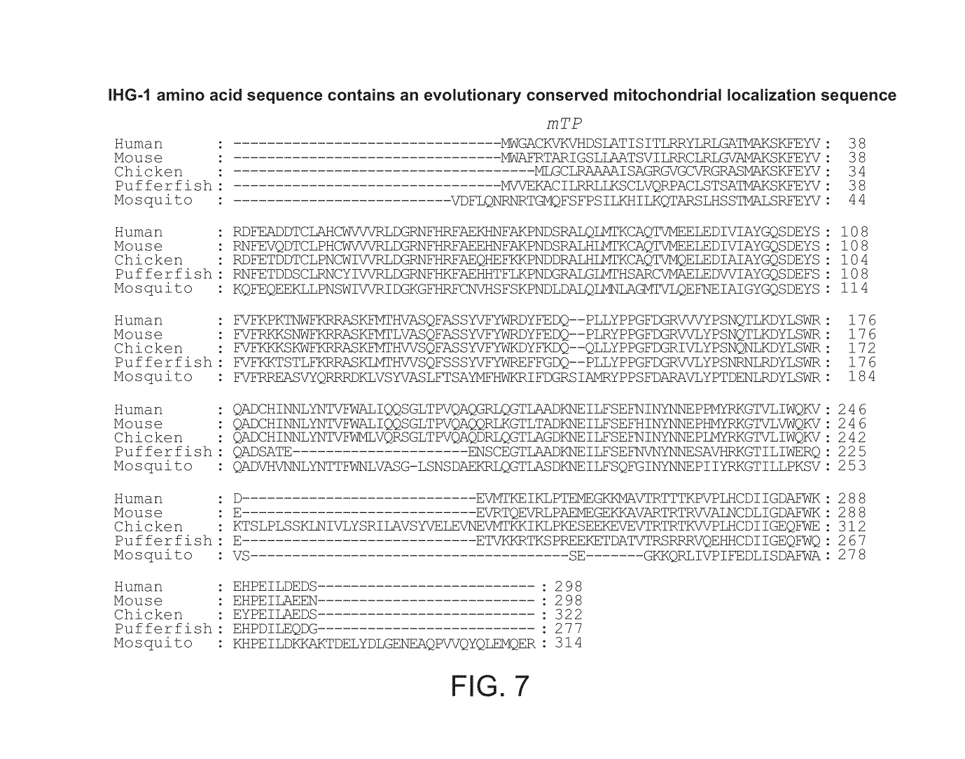

2. A protein as claimed in claim 1 having the sequence shown in FIG. 7 with a mutation in the region identified as mTP which results in the loss of the mitochondrial localisation signal.

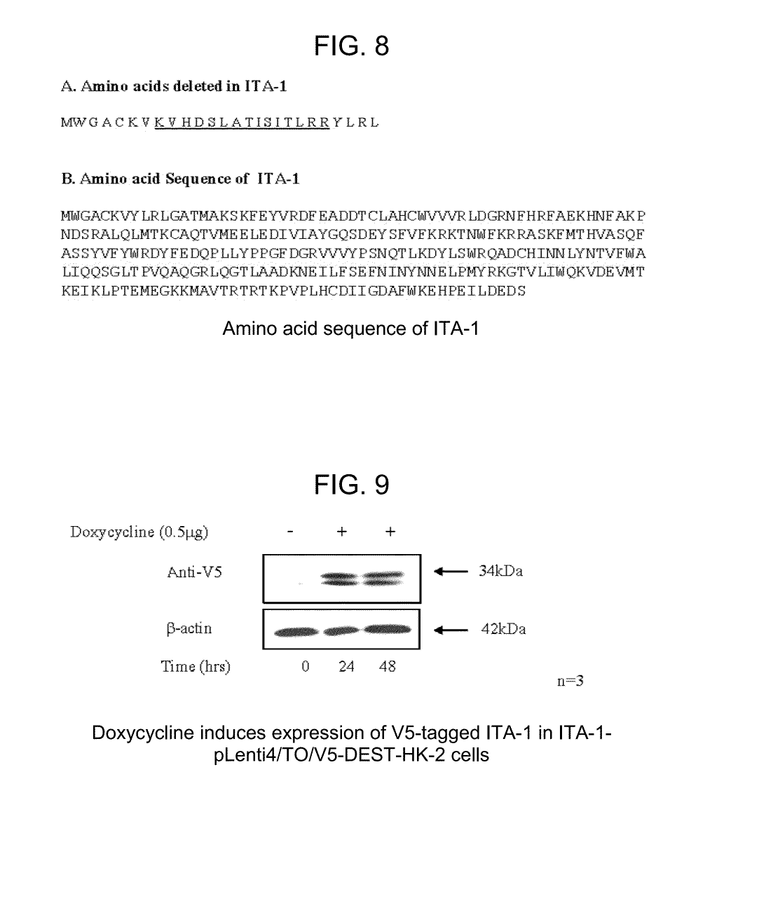

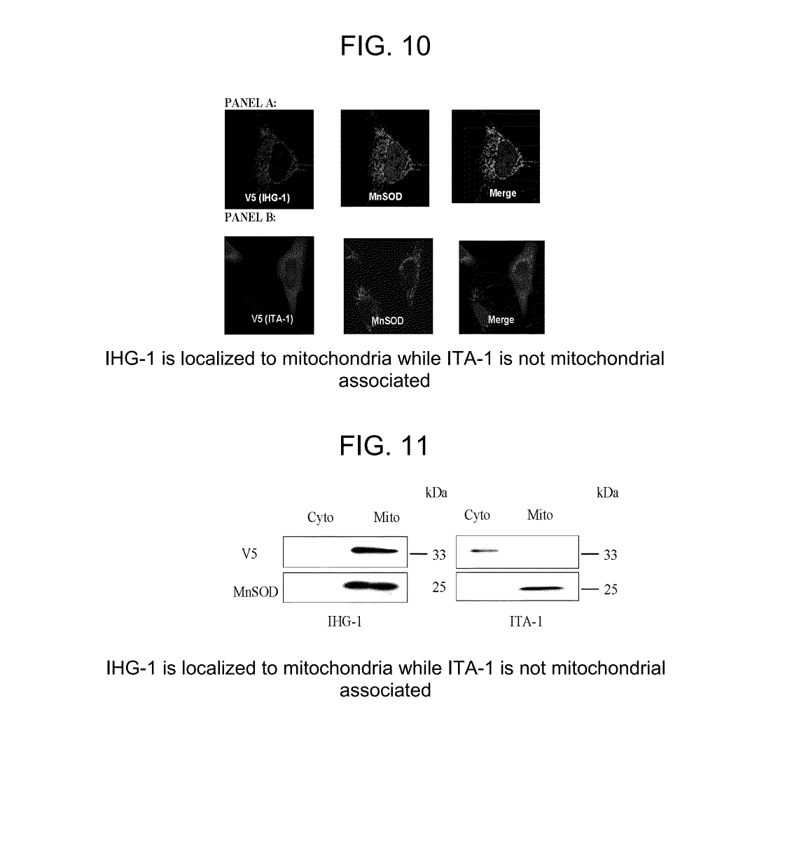

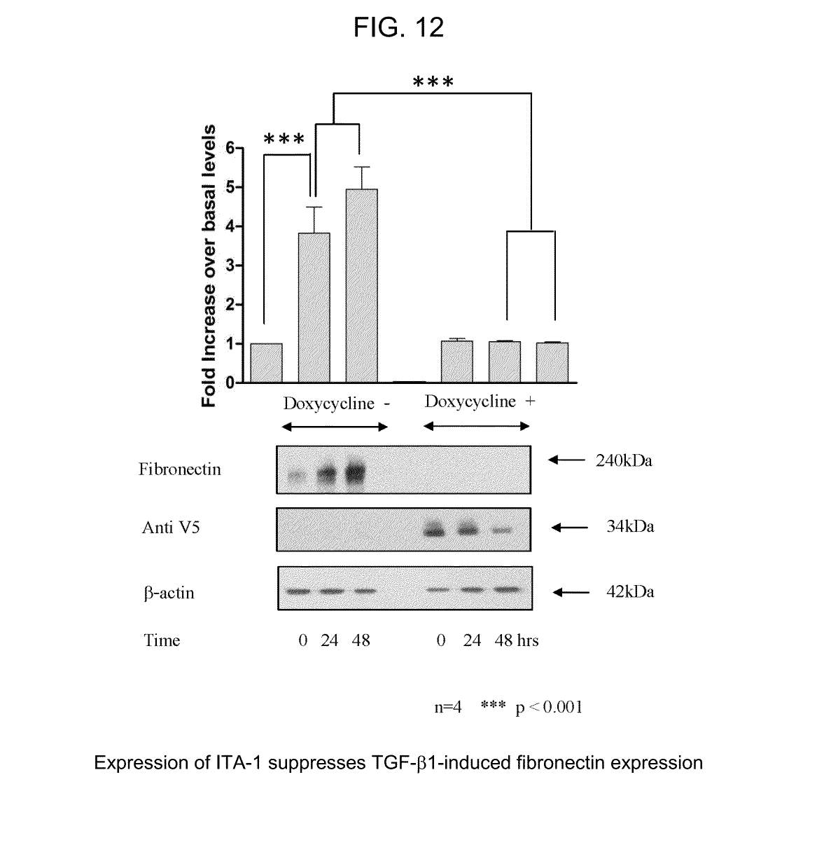

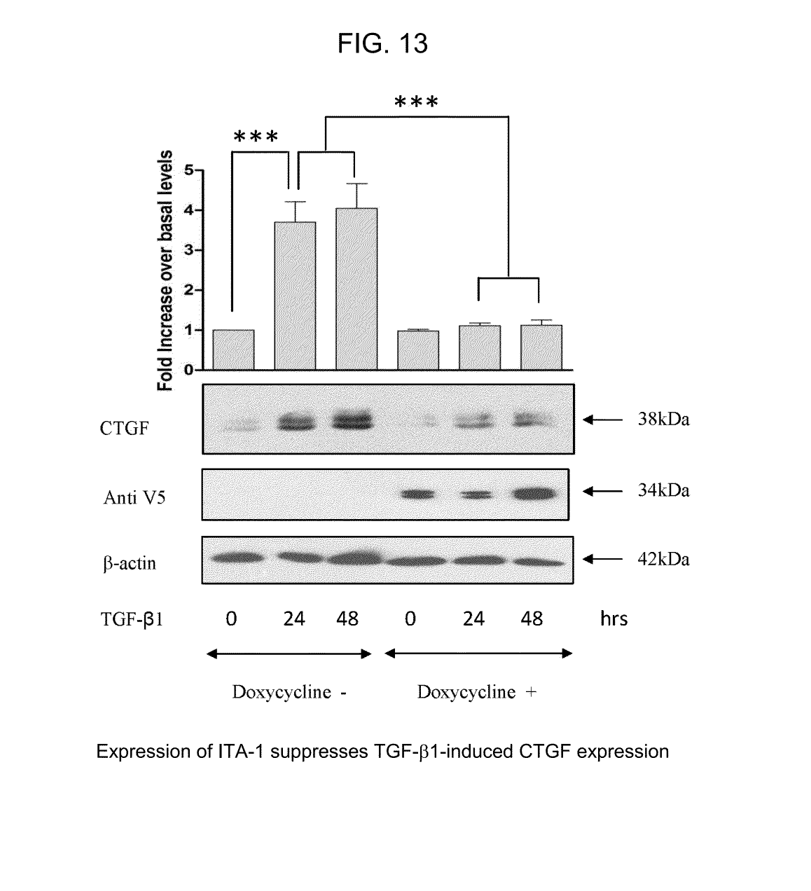

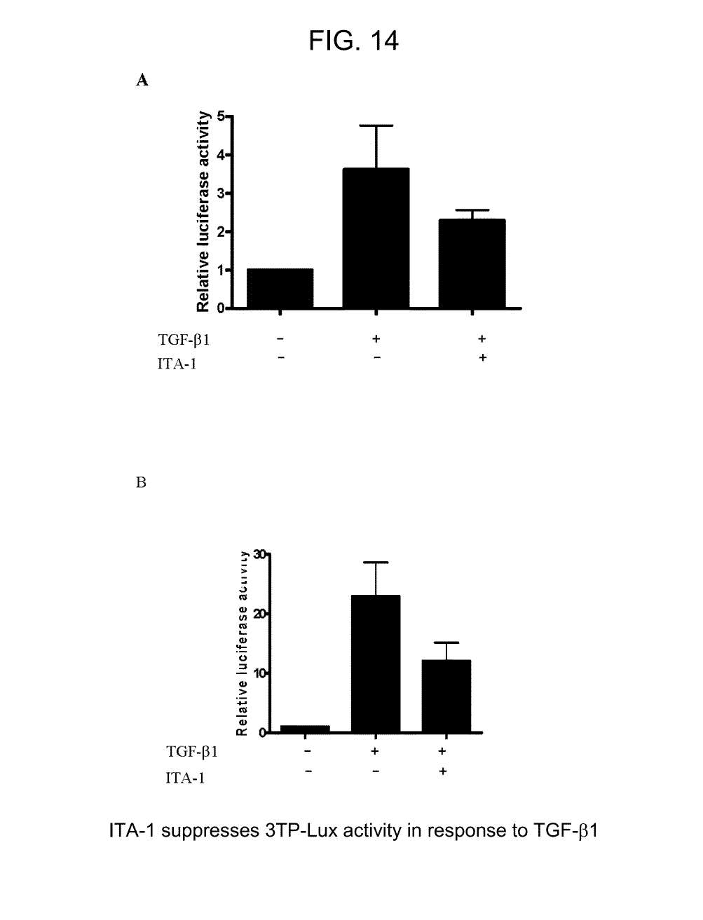

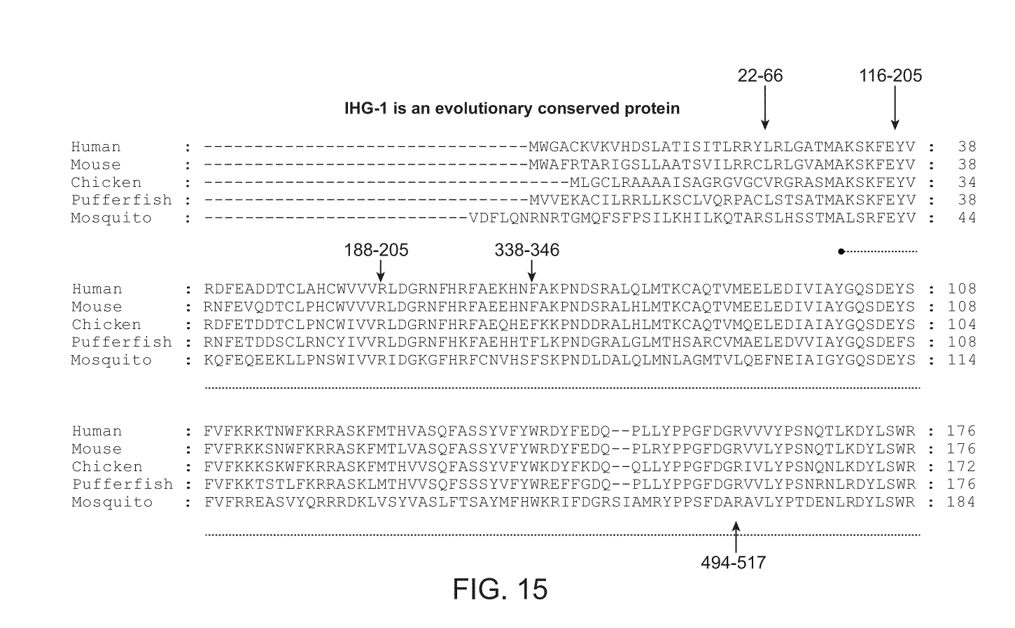

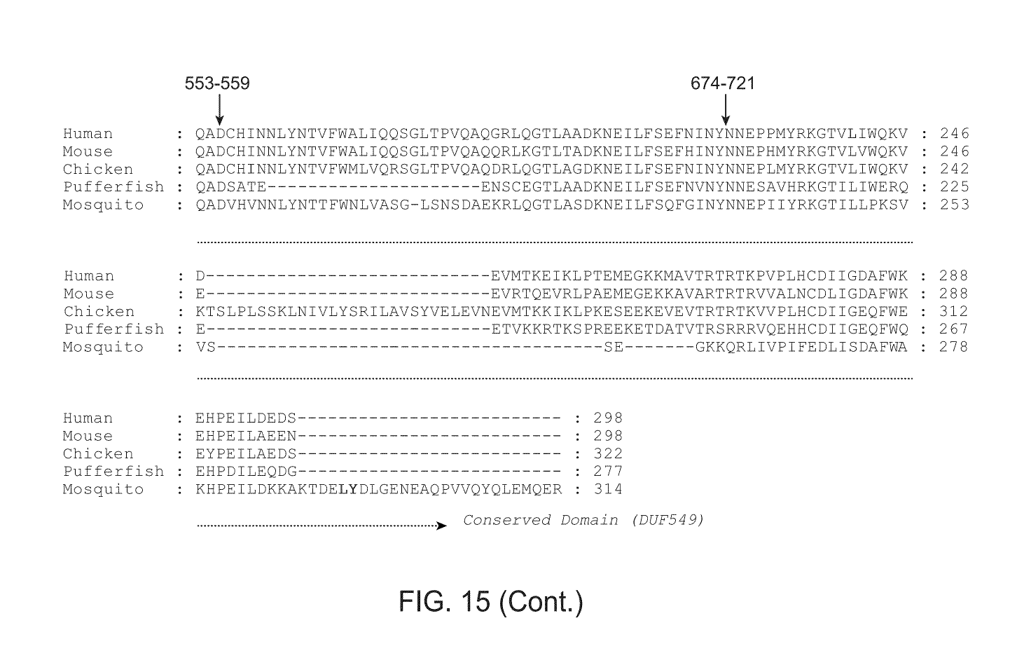

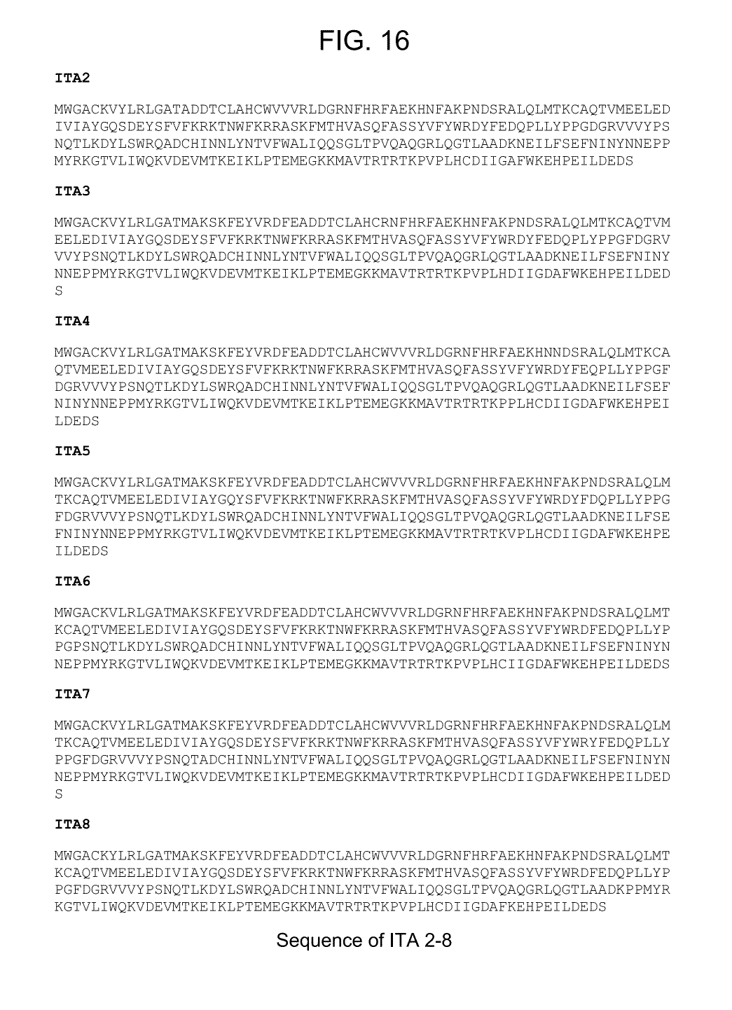

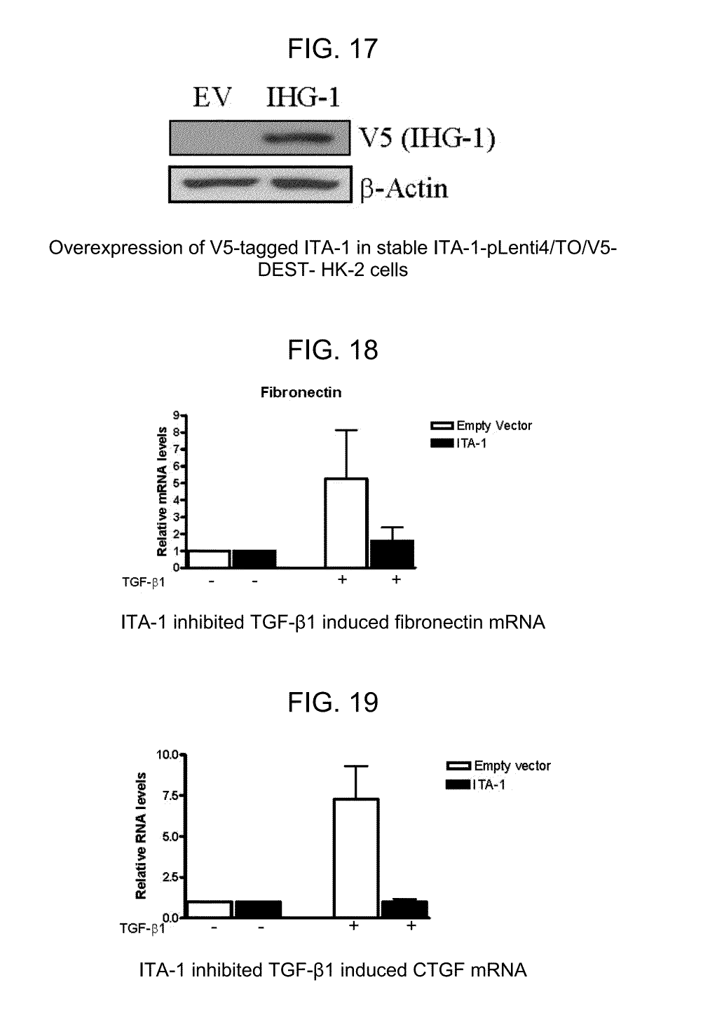

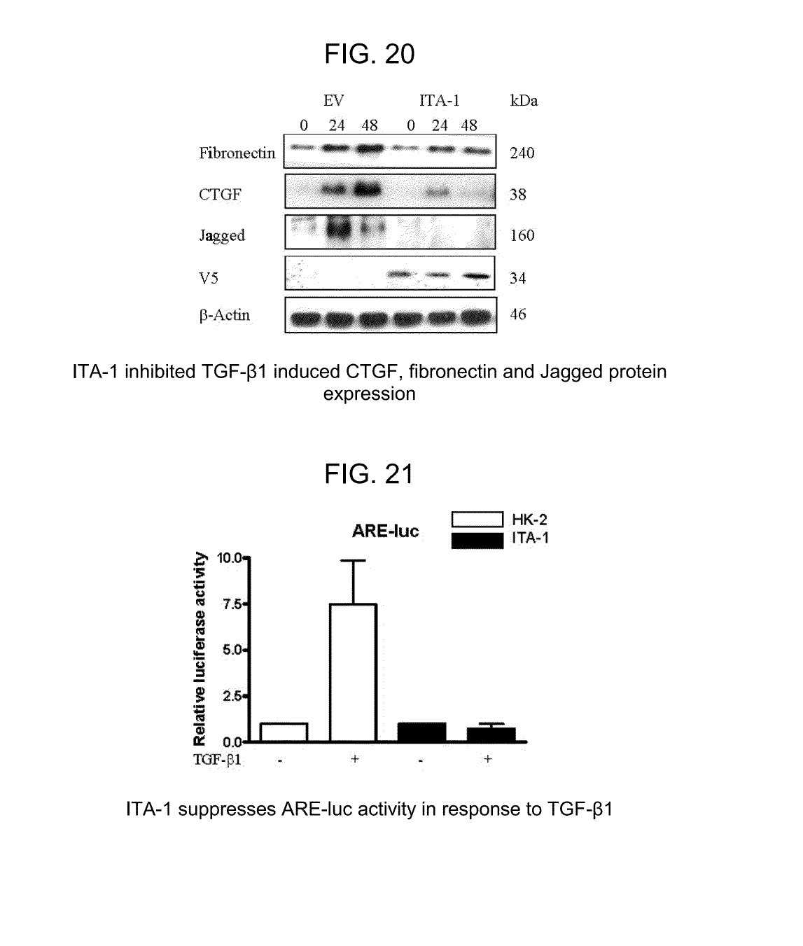

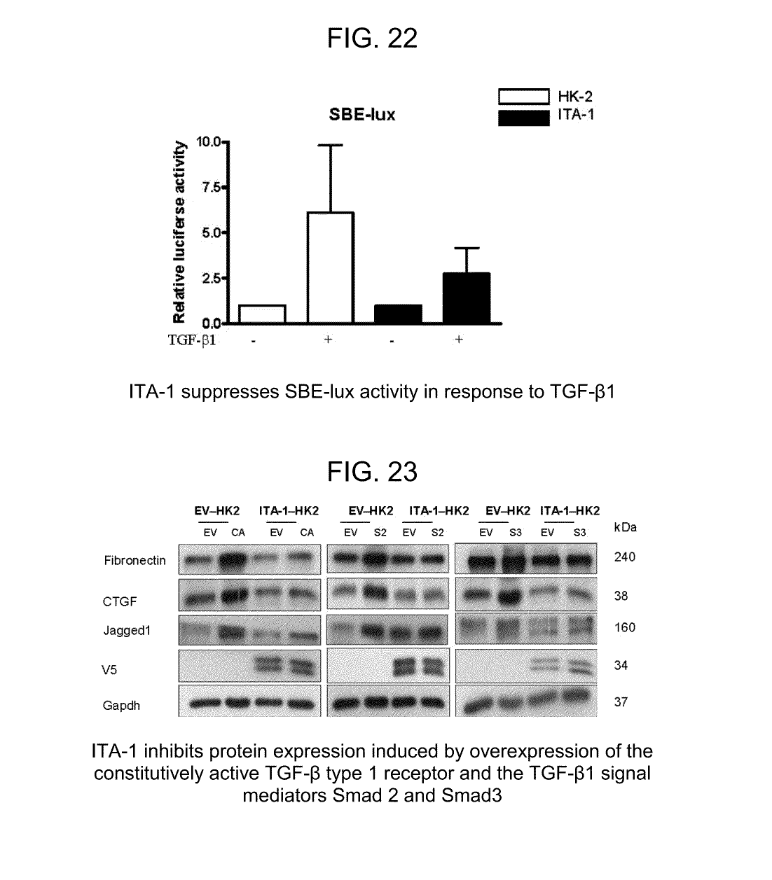

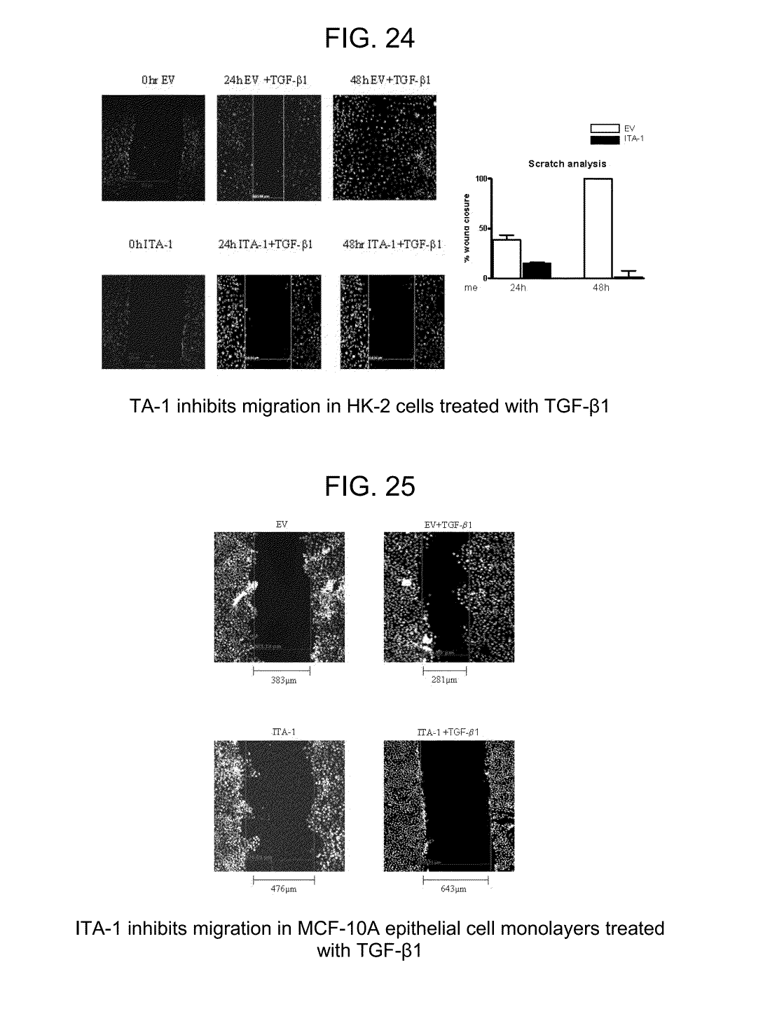

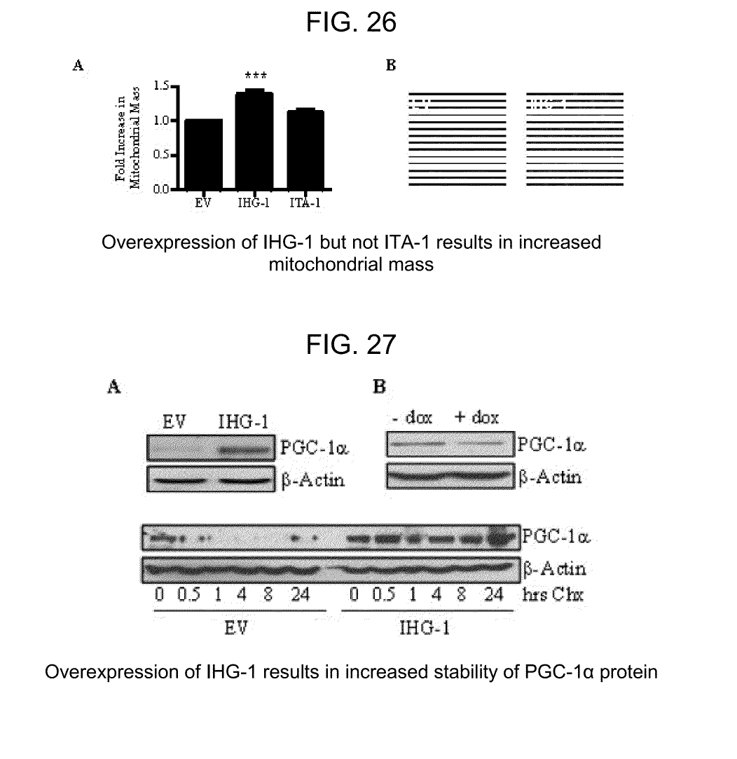

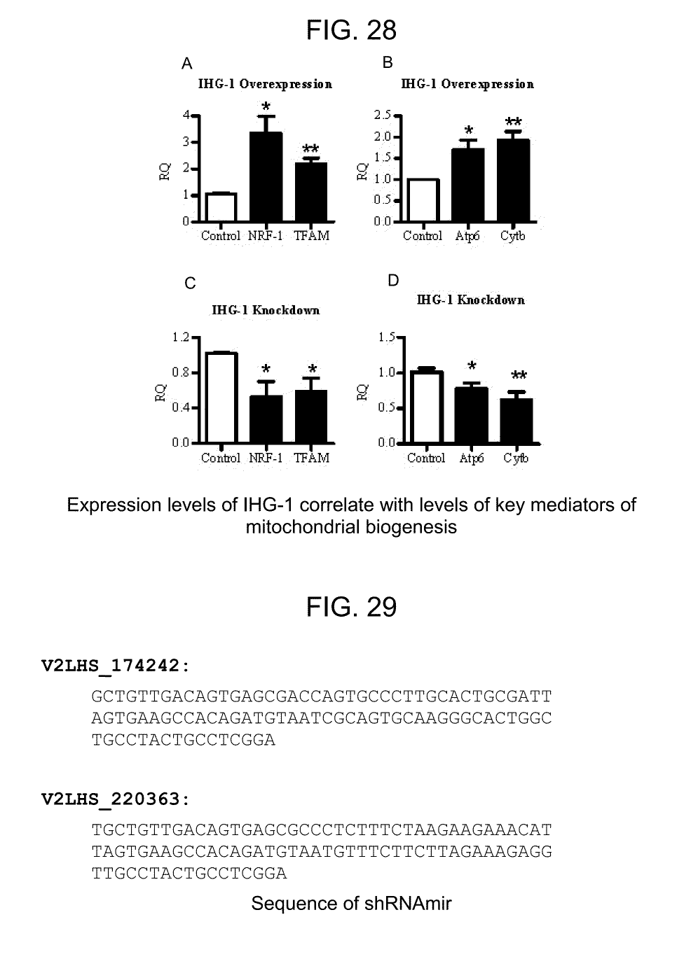

3. A protein as claimed in claim 1 or 2, wherein the mutation results in the loss or inactivation of the sequence shown in FIG. 8.

4. A protein for the treatment of fibrotic disease or for the treatment of a condition characterised by dysregulated cellular invasiveness, such as cancer, having a sequence selected from the group comprising the sequences shown in FIG. 16.

5. A peptide for the treatment of fibrotic disease or for the treatment of a condition characterised by dysregulated cellular invasiveness, such as cancer, derived from a protein as claimed in claim 1 or 4.

6. A peptidomimetic for the treatment of fibrotic disease or for the treatment of a condition characterised by dysregulated cellular invasiveness, such as cancer, based on a protein or peptide as claimed in claim 1 or 4.

7. A recombinant vector comprising a nucleotide sequence encoding a protein or peptide as claimed in claim 1 or 4.

8. A recombinant vector comprising a nucleotide sequence encoding peptide as claimed in claim 5.

9. A recombinant vector comprising a nucleotide sequence encoding peptidomimetic as claimed in claim 6.

10. A pharmaceutical composition comprising a protein as claimed in claim 1 or 4 or a polynucleotide encoding the protein, and a pharmaceutically acceptable carrier or excipient.

11. A pharmaceutical composition comprising a peptide as claimed in claim 5 or a polynucleotide encoding the peptide, and a pharmaceutically acceptable carrier or excipient.

12. A pharmaceutical composition comprising a nucleotide sequence encoding peptidomimetic as claimed in claim 6, and a pharmaceutically acceptable carrier or excipient.

13. A pharmaceutical composition comprising a vector is selected from the group comprising plenti6-V5-His, plenti4/TO/V5-DEST, pcDNA6-V5-His and a pharmaceutically acceptable carrier or excipient.

14. A method for studying TGF-β1 signalling for the discovery of a therapeutic targets, comprising contacting a sample with a protein of claim 1 or 4, or a peptide or peptidomimetic thereof.

15. A method for the treatment of a disease or condition mediated at least in part by Notch receptor activation, or in a method of reducing cell motility and/or invasiveness in a subject or patient in need thereof, comprising administering to the subject or patient a protein of claim 1 or 4, or a peptide or peptidomimetic thereof.
